(19) United States
(12) Patent Application Publication
(10) Pub. No.: US 2025/0180558 A1
(43) Pub. Date: Jun. 5, 2025

(54) BOOSTED IMMUNE MONITORING METHODS FOR ASSAYING ANTIGEN-SPECIFIC T CELL RESPONSES

(71) Applicants: INSTITUCIÓ CATALANA DE RECERCA I ESTUDIS AVANÇATS, Barcelona (ES); FUNDACIÓ PRIVADA INSTITUT DE RECERCA SOBRE IMMUNOPATOLOGIES-CAIXA IRSICAIXA, Badalona (ES)

(72) Inventors: Marta RUIZ RIOL, Tossa de Mar (ES); Christian BRANDER-SILVA, Tiana (ES); Alex OLVERA VAN DER STOEP, Vic (ES); Luis ROMERO MARTÍN, Badalona (ES)

(21) Appl. No.: 18/844,831

(22) PCT Filed: Mar. 9, 2023
(86) PCT No.: PCT/EP2023/055951

§ 371 Date: Sep. 6, 2024

Related U.S. Application Data
(60) Provisional application No. 63/317,973, filed on Mar. 9, 2022.

(51) Int. Cl. G01N 33/569

(52) U.S. Cl. CPC G01N 33/56972; G01N 2333/35; G01N 2333/54; G01N 2333/5406; G01N 2333/57

(57) ABSTRACT

The present invention relates to a method for assaying antigen-specific T-cell responses through boosted flow cytometry and boosted ELISPOT technologies that are based on the use of specific combinations of monoclonal antibodies to stain simultaneously cytokines that in their combination identify specific T-cell polarization profiles. The method allows the identification of Tfc CD8+ T cell responses unrecognized so far.

### FIELD OF THE INVENTION

The present invention relates to a method for assaying antigen-specific T cell responses based on boosted flow cytometry and boosted ELISPOT technology and the use of combinations of monoclonal antibodies against cytokines defining specific T-cell polarization profiles that are related with disease status.

### BACKGROUND OF THE INVENTION

Despite 90 years of vaccination and 60 years of chemotherapy, tuberculosis (TB) remains the world's leading cause of death from an infectious agent, exceeding human immunodeficiency virus/acquired immune deficiency syndrome (HIV/AIDS) for the first time. The World Health Organization (WHO) estimates that there are about 10.4 million new cases and 1.8 million deaths from TB each year. One-third of these new cases (about 3 million) remain unknown to the health system, and many are not receiving proper treatment.

Tuberculosis is an infectious bacterial disease caused by Mycobacterium tuberculosis (Mtb), which is transmitted between humans through the respiratory route and most commonly affects the lungs but can damage any tissue. One of the challenges of TB is that the pathogen persists in many infected individuals in a latent state for many years and can be reactivated to cause disease. The risk of progression to TB disease after infection is highest soon after the initial infection and increases dramatically for persons co-infected with HIV/AIDS or other immune-compromising conditions.

Treatment of TB disease requires multiple drugs for many months. These long drug regimens are challenging for both patients and health care systems, especially in low- and middle-income countries (LMICs), where the disease burden often far outstrips local resources. In some areas, the incidence of drug-resistant TB, requiring even longer treatment regimens with drugs that are more expensive and difficult to tolerate, is increasing.

Much progress has been made in TB control over the past several decades. Despite progress, major gaps persist. Although the Sustainable Development Goals (SDGs) seek to end the tuberculosis epidemic altogether, the decline in incidence has been disappointing. One of every three TB patients remains “unknown to the health system,” many are undiagnosed and untreated, and case detection and treatment success rates remain too low in the high-burden countries. Ominously, rates of multidrug-resistant (MDR) TB—defined as resistance to the two major TB drugs, isoniazid and rifampicin—are rising globally with the emergence of extensively drug-resistant (XDR) TB, resistant to many second-line drugs, as well as strains resistant to all current drugs. These are now primarily the result of transmission rather than inadequate treatment.

If the ultimate goal of controlling an infectious disease is to interrupt transmission, turning the tide on TB will require early and accurate case detection, rapid commencement of and adherence to effective treatment that prevents transmission, and, where possible, preventive treatment of latent TB.

Diagnosis in LMICs is made primarily by microscopic examination of stained smears of sputum of suspected patients; however, smear microscopy is capable of detecting only 50-60 percent of all cases (smear-positive). New diagnostic options that have emerged in recent years, such as the Xpert MTB/RIF test, a DNA amplification test that provides rapid and sensitive detection of TB and rifampicin resistance which allows to diagnose TB and identify MDR TB from sputum in less than two hours. However, this test is expensive and cannot be done at points of care. Zak D et al. (2016. The Lancet 387 (10035): 2312-22) have developed method that measures gene expression signatures in peripheral white blood cells that are elevated in healthy individuals with latent infection prior to their progression to active TB.

Still, there is a need for further diagnostic methods for TB which bring health care providers closer to TB control. More sensitive diagnostic methods which could discriminate between active and latent TB infections (ATBI and LTBI) would be desirable to properly adjust the treatment and achive said TB control.

Around 38 million people worldwide are presently infected with HIV. See UNAIDS, http://wwwunaids.org/, November 2021. HIV infection is currently one of the major public health challenges in the world.

A small percentage of HIV infected individuals (<1%, aka HIV controllers) naturally controls viral replication in the absence of HAART, likely by innate and adaptive immune mechanisms (Jacobs E, et al., J. Virol. 2017; doi:10.1128/jvi.02051-16, Côrtes F, et al., J Acquir Immune Defic Syndr. 2015; doi:10.1097/QAI.0000000500). The detailed understanding of this natural immune-mediated control is critical for the development of effective immune strategies for prevention and cure of HIV infection (Côrtes, 2015, supra). Specific HLA class I genotypes have shown some degree of association with HIV disease progression by inducing efficient cytotoxic CD8+ T-cell responses (CTL or Tc)³, but this only account for 10-15% of the infection course variability in untreated hosts (Jones R, et al., J. Clin. Invest. 2016; doi:10.1172/JCI80566, Fellay J, et al., Science 2007; 80, doi:10.1126/science.1143767). Moreover, the relative contribution of different CTL specificities to overall virus control remains controversial due to its high diversity among HIV controllers (Pereyra F, et al., J. Infect. Dis. 2008; doi:10.1086/526786, Sáez-Cirión A, et al., J. Immunol. 2009; doi:10.4049/jimmunol.0803928). In addition to cellular immunity, humoral immune correlates of controlled HIV disease have also been explored. In particular, passive immunization with broadly neutralizing antibodies (bnAb) has shown compelling clinical efficacy, especially when using structurally modified antibodies (Mendoza P, et al., Nature 2018; 561). These studies clearly demonstrate that neutralizing, as well as non-neutralizing antibodies to HIV can trigger additional antiviral effector functions, involving Fc-mediated mechanisms such as antibody-dependent cell-mediated cytotoxicity (ADCC) (Chen X, et al., Front. Immunol. 2018; doi:10.3389/fimmu.2018.02322). However, naturally induced neutralizing antibodies seem to have a limited capacity to control chronic viral replication, and broadly neutralizing antibody responses are only elicited in a minority of infected individuals; furthermore, their presence is often associated with increased HIV replication and viral diversity (Overbaugh J, et al., Cold Spring Harb. Perspect. Med. 2012; doi:10.1101/cshperspect.a007039, Su B, et al., Front. Immunol. 2019; doi:10.3389/fimmu.2019.02968). Aside from affinity maturation that can give raise to bnAbs, the mechanisms that lead to the induction and maintenance of humoral responses with effector functions are less well understood.

While it is widely accepted that effective HIV control requires the elicitation of both, strong cellular and humoral adaptive responses, the nature of the most appropriate T cell population to exert direct antiviral activity and support antibody production remains elusive. However, emerging data strongly indicate that it may be a question of specificity as well as of the polarization of the effector profiles by such T-helper cells (Th) that drive this combined antiviral immunity. Pathogen-specific memory T-cells can be found in different Th subsets that can have different, or even divergent effector functions (Sallusto F, Annu. Rev. Immunol. 2016; 34:317-334). While much is known about CD4 T-cells and their differentiation and polarization into different Th subsets (such as Th1, Th2, Thx-like populations) and divergent effector function profiles, such classification is less established for CD8+ T cells. Still, CD8+ T cells have been recently reported to also differentiate into distinctly polarized subsets, characterized by effector functions other than their classical cytotoxic capacity, and have been implicated in different immune settings, including settings beyond HIV infection (Sallusto, 2016, supra, Valentine K, et al., Front. Immunol. 2019; doi:10.3389/fimmu.2019.01322). For instance, CD8+ subtypes with an unusual Th2-like phenotype have been associated with effective immune responses to Mycobacterium tuberculosis (Mtb).

Based on the broad spectrum of effector function profiles (mostly defined by cytokine production signatures), T cell polarization is widely classified in three major types: Type 1 (CD4+ Th1 and CD8+ Tc1 subsets, characterized by the secretion of IFN-γ, IL-2 and TNF-α), Type 2 (CD4+ Th2 and CD8+ Tc2 subsets, which mainly produce IL-4, IL-5, IL-10 and IL-13) and Type 3 (CD4+ Th17 and CD8+ Tc17 subsets, with IL-17 and IL-22 as signature cytokines) (Annunziato F, et al., J. Allergy Clin. Immunol. 2015; 135:626-635). This T-cell subset diversity is completed by regulatory T-cells (Treg, either CD4+ or CD8+ Treg subsets, which produce IL-10 and TGF-β), a number of less characterized, rarer subsets (Th22 CD4+ or CD8+ subsets, defined by TNF-α, IL-2 and IL-22 production; CD4+ and CD8+ Th9 characterized by production of IL9, and other emerging subsets)(Chen J, et al., DNA Cell Biol. 2019; 38(10):1040-1047, Jia L, et al., Adv Exp Med Biol. 2014; 841:209-230). One of the more recently identified additional T cell subsets are the follicular T-cells. Follicular T helper-cells (Tfh) have been shown to play an important role in the development of humoral immunity (Joosten S, et al., Hum. Immunol. 2008; 69:760-770). Located at the edge of the T-cell zone in secondary lymphoid organs, activated naïve CD4+ pre-Tfh cells start expressing CXCR5, PD-1 and ICOS and migrate to the germinal center of the follicles (Song W, et al., Immunol. Rev. 2019; 288:85-96). There, they interact with B-cells, supporting their proliferation, isotype class switching and antibody affinity maturation through serial somatic hypermutations. CD4 Tfh-functionality is related to IL-4 and IL-21 secretion, which is necessary for the establishment of antigen-specific, long-lived plasma cells and circulating memory B-cells (Koutsakos M, et al., J. Immunol. 2019; 202:360-367). Although it was initially thought that this function is reserved to CD4+ T-cells, there is growing evidence that CXCR5 expressing CD8+ T-cells also localize in, or proximal to, B-cell follicles. These CD8+ T follicular cytotoxic cells (Tfc) have been reported to have a self-renewal and less-exhausted profile, and to keep their cytotoxic functionality to eliminate infected cells while regulating the antibody response (Yu D, et al., Trends Immunol. 2018; 39:965-979). These findings are in line with prior observations, where CD8 Tfc were related to B cell isotype switching and superior control of viral replication.

The association between type 1 responses and reduced HIV viremia has been demonstrated for CD8+ and CD4+ T-cells. Despite being postulated many years ago, the importance of other T-cell subsets exerting alternative effector functions (not mediated by IFN-7) in HIV pathogenesis remains less well studied (Walker B, et al., Cold Spring Harb. Perspect. Med. 2012; doi:10.1101/cshperspect.a007054, Johnson S, et al., J. Virol. 2015; doi:10.1128/jvi.00438-15). Several aspects might be responsible for this gap in our knowledge, including the small size of some subsets in peripheral blood, their heterogeneity and plasticity or the severe CD4+ T-cell depletion suffered during acute HIV infection (Crotty S, et al., Cold Spring Harb. Perspect. Biol. 2018; 10:a032102). In the early 1990's, the CD4+ Th1/Th2 balance was considered a hallmark of HIV disease progression, although that view was not uniformly shared (Graziosi C, et al., Science 1994; 80:265, Clerici M, et al., Immunol. Today 1993; 14). To date, while HIV-specific type 2 and type 3 responses remain poorly understood, the relevance of CD4+ Th2 and Th17 in HIV disease control and vaccine response begins to emerge (Sauce D, et al., Sci. Rep. 2016; doi:10.1038/srep28129). Conversely, HIV-specific CD8+ Tc2 responses are associated with reduced cytotoxic activity while the preservation of tissue-resident CD8+ Tc17 during HIV infection is important to regulate immune activation (González S, et al., Front. Immunol. 2017; 8:1-9, Perdomo-Celis F, et al., Front. Immunol. 2017; 8:1-13). Regulatory CD4+ T-cells have been associated with weaker HIV-specific cellular immune responses and shown to suppress HIV-induced immune hyperactivation and thus limit infection of conventional CD4+ T-cells (Hasenkrug K, et al., PLoS Pathog. 2018; 14:1-22, Fenoglio D, et al., J. Allergy Clin. Immunol. 2018; 141:2220-2233.e4). CD4+ Tfh-cells have been reported to have different HIV antigen specificities compared to conventional CD4+ Th1 cells and to be needed for providing help to differentiate and mature the HIV-specific B-cell response, while supporting CTL activity (Locci M, et al., Immunity 2013; 39:758-769, Martin-Gayo E, et al., JCI Insight 2017; 2:1-17, Schultz B, et al., Immunity 2016; 44:167-178). On the other hand, Tfh-cells seem to be highly permissive to HIV entry and constitute a significant proportion of the latent viral reservoir and expansion of this cell subset is associated with disease progression (Aid M, et al., Front. Immunol. 2018; doi:10.3389/fimmu.2018.00895, Kohler S, et al., J. Immunol. 2016; doi:10.4049/jimmunol.1502174, Miles B, et al., Trends Microbiol. 2016; doi:10.1016/j.tim.2016.02.006, Lu J, et al., J. Infect. Public Health 2018; 11:685-690).

Less is known for CD8+ Tfc cells in HIV infection, with some studies reporting a higher ex vivo cytotoxic activity compared to non-Tfc CD8+ T cells (Petrovas C, et al., Sci. Transl. Med. 2017; doi:10.1126/scitranslmed.aag2285). This is in line with CD8+ T cells of Tfc and Tc2-like phenotypes that mediate lower viral loads in animal models of chronic infection, including LCMV infected mice and SIV infected macaques (He R, et al., Nature 2016; doi:10.1038/nature19317, Mylvaganam G, et al., Proc. Natl. Acad. Sci. USA 2017; doi:10.1073/pnas.1621418114). These observations indicate that CD8+ Tfc with increased cytotoxic capacity and localized to lymph node follicles are critical in limiting the viral reservoir in CD4+ Tfh cells and provide additional help for the maturation of the humoral immunity in the follicles.

The above described diversity of T cell profiles and functional polarization is in stark contrast to the limited identification methodologies available to date to identify, characterize and quantify T cell responses. As a consequence, standard techniques do (i) miss such, aforementioned unconventional T cell differentiation profiles (Guardo A, et al., AIDS 2015; 29, Ruíz-Riol M, et al., J. Infect. Dis. 2015; doi:10.1093/infdis/jiu534) and (ii) are unable to provide further insights into disease status that is reflected by the polarization of the specific T cell response. Therefore, there is an urgent need in the art for better tests, such as the novel flow cytometry and boosted ELISPOT assays described herein, capable of identifying the broadest T-cell polarization profiles of antigen-specific T cells in a specific and sensitive manner.

### SUMMARY OF THE INVENTION

In a first aspect, the present invention relates to a method for assaying polarized T cell responses specific against an antigen in a subject which comprises:
(i) contacting a biological sample comprising T cells from the subject with a composition comprising the antigen and
(ii) determining the levels of a plurality of cytokines and/or chemokines produced by the T cells in the sample by boosted flow cytometry and/or boosted ELISPOT.
wherein a combination of antibodies against the plurality of cytokines and/or chemokines is used and the plurality of cytokines and/or chemokines is related to a specific T-cell polarization profile,
wherein the antigen is at least one antigen from M. tuberculosis.

In another aspect, the invention relates to a kit comprising reagents for the detection of one or more cytokines comprising IFN-γ, IL-2, IL-4, IL-5, IL-9, IL-10, IL-13, IL-17, IL-21, IL-22, TGF-β, CXCL-13 and TNF-α.

In another aspect, the invention relates to the use of the method of the first aspect for the diagnosis of tuberculosis.

### BRIEF DESCRIPTION OF THE DRAWINGS

FIG. 1. The Boosted flow assessment ability to capture more powerful M. Tb infection compared with other assessments (50% Gold Standard Quantiferon IGRA (QFN) vs 100% boosted (5 ug of protein stimulation) and 15% Tuberculin Skin Test vs 100% LTBI (5 ug of protein stimulation) in the ATBI. Chi-square performed for groups comparisons. In the graph is indicated the percentage of patients that respond to the indicated test with a positive response. P<0.05 are consider significant. P<0.0001=****, P<0.001=***, P<0.01=**.

FIG. 2. While ATBI predominately generates Th1-like and Th17-like profiles, LTBI generates specific M.Tb responses with Th2-like and Tfh effector functions. Chi-square performed for groups comparisons. In the graph is indicated the percentage of patients that make a positive response with a specific T cell profiles measured by Boosted flow in response to ESAT-6, CFP-10 or the combination of both proteins (5 ug stimulation). P<0.05 are consider significant. P<0.0001=****, P<0.001=***, P<0.01=**.

FIG. 3. Gating strategy for boosted flow screening. Viable CD3+ cells were gatted followed by CD4 and CD8 gatting. Boosted flow channels are shown for CD4+ cells (A) and CD8+ cells (B). Specific CD4 T cell (A) and CD8 T cell responses (B) in the presence of anti-CD3/CD28 polyclonal stimulation (positive control) or in the absence of specific T cell stimulator (negative control).

FIG. 4. Correlation between IL-4/IL-21 responses measured by boosted flow and ELISPOT on total PBMCs upon HIV-peptide pool specific stimulation (non-parametric Spearman r test).

FIG. 5. HIV protein-specific polarized T-cell responses. Magnitude of the polarized responses in CD4+ T-cells (A) and CD8+ T-cell (B). Controllers are indicated in purple dots, and non-controllers by light blue dots. Significate p-values are highlighted in color, purple if responses were higher in controllers and light blue if responses were higher in non-controllers. The tables below represent the number of pools contained in each protein-screening with the number of peptides in each pool in brackets. Statistical significance was evaluated by non-parametric Mann-Whitney test (B).

FIG. 6. Lack of significant correlation between HIV-specific CD8+ Tfc-like responses and humoral responses for (a) neutralization and (b) ADCC. P stated for Present. A, for Absent.

FIG. 7. Boosted Flow screening of specific-T-cell responses against the entire HIV proteome in a cohort of 15 HIV+ controllers (C, purple) and 14 HIV+ Non-controllers (NC, light blue) revealed a highly-diverse set of polarized T-cell responses in terms of (A) number of responders (B) percentage of circulating HIV-specific T-cells (upper panels) and number of reactive pools (lower panels) for CD4 and CD8 T cells, respectively. (C) Negative correlation between plasma viral load and CD8+ Tfc-like T cell responses to HIV measured in terms of breadth (left) and magnitude (right). (D) Capacity of total PBMCs to secrete T-follicular associated cytokines IL-21 (left scatter plot), IL-4 (middle scatter plot) and IL-21/IL-4 simultaneously (right scatter plot) upon antigen unspecific PHA stimulation stratified by the presence (left bar) or the absence (right bar) of HIV-specific circulating Tfc-like responses. Statistical significance of differences in frequency of responders was evaluated by Chi-square test, between group medians by non-parametric Mann-Whitney test and correlation between Tfc-like responses and plasma viral load by Spearman correlation and its correspondent p-value. Statistical significance was set at p<0.05.

FIG. 8. Comprehensive assessment of HIV protein-specific, polarized T-cell responses. Boosted flow cytometry-based screening of HIV protein-specific T-cell responses reveals antigen-dependent, different polarized profiles in both CD4+ T-cells and CD8+ T-cells. The percentage of responders to each of the HIV-1 derived proteins is indicated in stacked bars for CD4+ (A) and CD8+ (B) T-cells. Statistically significant (p<0.05) differences in protein-specific frequency of response between HIV+ controllers (C) and HIV+ non-controllers (NC) are indicated. Statistical significance was evaluated by Chi-square and statistical significance set at p<0.05.

FIG. 9. Memory phenotype of the HIV-specific polarized T-cell responses. The frequency of cells expressing memory markers CD45RA and CCR7 was evaluated in both, HIV-specific polarized CD4+ and CD8+ T-cells. The percentage of basal, Th1/Tc1, Th2/Tc2 and Tfh/Tfc-like cells classified as TCM (CD45RA− CCR7+), TEM (CD45RA− CCR7−), TEMRA (CD45RA+ CCR7−) and TNaive (CD45RA+ CCR7+) are shown in pie charts. Statistical significance of the differences between HIV-specific memory phenotype in HIV+ controllers (C, left) and HIV+ Non-controllers (NC, right) was evaluated by non-parametric Mann-Whitney test, statistical significance was set at p>0.05, only significant results are shown.

FIG. 10. Link between HIV-specific CD8+ Tfc-like responses and humoral responses. (A) Samples were stratified by the presence (n=17) or the absence (n=13) of HIV-specific CD8+ Tfc-like responses and titers of IgA, IgG and IgM were compared, as well as the IgA/IgM, IgG/IgM and IgG/IgA ratios. (B) Correlation between anti-HIV-1NL4-3 (left) and HIV-1BaL (right) IgM titers and the magnitude (left) and breadth (right) of the CD8+ Tfc-like response. (C) Correlation between anti-HIV-1_{NL4-3} (left) and HIV-1_{BaL} (right) IgG titers with CD8+ Tfc-like breadth. (D) Correlation between anti-HIV-1_{NL4-3} (left) and HIV-1_{BaL} (right) IgG/IgM ratio with CD8+ Tfc-like breadth. (E) Correlation between anti-HIV-1_{NL4-3} (left) and HIV-1_{BaL} (right) IgG/IgA ratios and the magnitude (left) and breadth (right) of the CD8+ Tfc-like response. Statistical significance of the comparison was performed by non-parametrical Mann-Whitney test while non-parametric Spearman r correlations were applied.

### DETAILED DESCRIPTION OF THE INVENTION

At present, there is no test that can discriminate between active and latent stages of TB. The inventors have surprisingly found that boosted flow cytometry outperforms prior art tests for both active and latent TB diagnosis: boosted flow is significantly more powerful to detect active M.tb. infection compared to Gold Standard Quantiferon IGRA (37% vs 100%) and Tuberculin Skin Test (TST) (50% vs 100%). With the present invention, different polarization profiles are assessed at the same time, in the same sample, without the need for additional parallel tests, and allowing the breakdown by T-cell polarization profiles to discriminate between active and latent infection. This is particularly relevant and useful for children, elder people and immunocompromised patients, as for example patients coinfected with HIV-1, where TB is an important cause of mortality, or patients under immunosuppressive treatment. The ability of the claimed invention for discriminating between active and latent stages of TB disease is an important added value compared with the gold standard assays in the market, very relevant for the treatment adequacy to reduce the multidrug resistance that has become a worldwide challenge.

### 1. Definitions of General Terms and Expressions

The term “AIDS”, as used herein, refers to the symptomatic phase of HIV infection, and includes both Acquired Immune Deficiency Syndrome (commonly known as AIDS) and “ARC,” or AIDS-Related Complex (Adler M, et al., Brit. Med. J. 1987; 294: 1145-1147). The immunological and clinical manifestations of AIDS are well known in the art and include, for example, opportunistic infections and cancers resulting from immune deficiency.

The term “antigen”, as used herein, refers to a molecule recognized by the products of the adaptative immune response (specifically, recognized by specific T cell receptor (TCR) and B cell receptors (BCR, or antibodies). Antigens include but are not limited to cells, cell extracts, proteins, polypeptides, peptides, polysaccharides, polysaccharide conjugates, peptide and non-peptide mimics of polysaccharides and other molecules, small molecules, lipids, glyco lipids, carbohydrates, viruses and viral extracts and multicellular organisms such as parasites and allergens. The term antigen broadly includes any type of molecule which is recognized by a host's immune system as such including, but not limited to, pathogen-derived antigens, self-antigens, allergens, and toxins.

The term “antiretroviral therapy” or “ART”, as used herein, refers to the administration of one or more antiretroviral drugs (i.e., HIV antiretrovirals) to inhibit the replication of HIV. Typically, ART involves the administration of at least one antiretroviral agent (or, commonly, a cocktail of antiretrovirals) such as nucleoside reverse transcriptase inhibitor (e.g., zidovudine (AZT, lamivudine (3TC) and abacavir), non-nucleoside reverse transcriptase inhibitor (e.g., nevirapine and efavirenz) and protease inhibitor (e.g., indinavir, ritonavir and lopinavir).

The term “antibody”, as used herein, refers to an intact immunoglobulin of any isotype, or a fragment thereof that can compete with the intact antibody for specific binding to the target antigen, and includes, for instance, chimeric, humanized, fully human, non-human, and bispecific antibodies. An antibody is a species of an antigen binding protein. An intact antibody will generally comprise at least two full-length heavy chains and two full-length light chains, but in some instances can include fewer chains such as antibodies naturally occurring in camelids which can comprise only heavy chains. Antibodies can be derived solely from a single source, or can be “chimeric” that is, different portions of the antibody can be derived from two different antibodies as described further below. The antigen binding proteins, antibodies, or binding fragments can be produced in hybridomas, by recombinant DNA techniques, or by enzymatic or chemical cleavage of intact antibodies. Unless otherwise indicated, the term “antibody” includes, in addition to antibodies comprising two full-length heavy chains and two full-length light chains, derivatives, variants, fragments, and muteins thereof, which may attach to the cytokines of the invention.

The term “M. Tb treatment or therapy”, as used herein, refers to the treatment received by subjects suspected or diagnosed of having M. tuberculosis (M. Tb) infection. For latent TB (LTBI), treatment is usually prescribed if there is a risk of developing active TB (ATBI) and most common treatment comprises only two types of drugs. For active tuberculosis, antibiotics are prescribed for at least six to nine months. Particularly for drug-resistant strains, the treatment will require several drugs at once, the most common medications being isoniazid, rifampicin, ethambutol, pyrazinamide and a combination of fluoroquinolones other injected antibiotics, such as amikacin or capreomycin, which are generally used for 20 to 30 months. Because some types of TB are developing resistance to these medications as well, some additional drugs might be added to therapy to counter drug resistance, such as bedaquiline or linezolid.

The term “multidrug resistance (MDR)”, as used herein, refers to the insensitivity or resistance of a microorganism to the administered antimicrobial medicines (which are structurally unrelated and have different molecular targets) despite earlier sensitivity to it.

The term “biological sample comprising T cells”, as used herein, refers to tissues or bodily fluids removed from a mammalian subject, and which contain T cells. In some embodiments, the T cells are isolated from the sample prior to their exposure to the antigen(s) of interest. The term “isolated” with respect to T cells refers to cell population preparation in a form that results in a cell population that has at least 70%, 80%, 90%, 95%, 99%, or 100% T cells. In some embodiments, a desired cell population is isolated from other cellular components, in some instances to specifically exclude other cell types that may interfere with the study of the cells in isolation. However, the “isolated” cell population may incorporate additional cell types that are necessary for cell survival or to achieve the desired results provided by the invention. For example, antigen presenting cells, such as monocytes (macrophages) or dendritic cells, may be present in an “isolated” cell population of T cells or added to a population of isolated T cells for generation of regulatory T cells. In some embodiments, these antigen presenting cells may be activated monocytes or dendritic cells. Cell populations comprising T cells for use in the methods of the invention may be isolated from a biological sample taken from a mammalian subject. The sample may originate from a number of sources, including, but not limited to peripheral blood, leukapheresis blood product, apheresis blood product, bone marrow, thymus, tissue biopsy, tumor, lymph node tissue, gut associated lymphoid tissue, mucosa associated lymphoid tissue, liver, sites of immunologic lesions (e.g., synovial fluid), pancreas, and cerebrospinal fluid.

The term “boosted flow cytometry”, as used herein, refers to an assay based on the detection of cells secreting simultaneously several cytokines and detecting these cytokines in the same channel of the flow cytometer so that the detected signals are additive across the detected cytokines. “Boosted flow cytometry” thus leads to an increased sensitivity and allows to cover a vastly larger set of effector functions than standard assays.

The term “boosted ELISPOT”, as used herein, refers to an assay based on the detection of cells producing several cytokines by using several cytokine-specific antibodies which are labelled (for instance, but not limited to enzymatic label like AP or fluorescent labels) for detection and quantification of cells producing one or several of these cytokines. The “boosted ELISPOT”, thus allows to detect a wider set of effector functions than standard, generally monofunctional, ELISPOT assays and provides a higher sensitivity than standard assays since the signals stemming from the individual cytokines produced in a single cell all add up to form a spot.

The term “CD4+ T cells”, as used herein, refers to T cells presenting the CD4 molecule (Cluster of Differentiation 4 molecule) on their surface. The term refers to T helper cells, which either orchestrate the activation of macrophages and CD8+ T cells (Th-1 cells), the production of antibodies by B cells (Th-2 cells) or which have been shown to play an essential role in autoimmune diseases (Th-17 cells). In addition, the term “CD4+ T cells” also refers to other CD4 expressing T cell populations, including but not limited to regulatory CD4+ T cells (Treg), which represent approximately 10% of the total population of CD4+ T cells. Regulatory T cells play an essential role in the dampening of immune responses, in the prevention of autoimmune diseases and in oral tolerance.

The term “CD8+ T cells”, as used herein, refers to T cells expressing the CD8 glycoprotein on their surface, wherein the CD8 (Cluster of Differentiation 8 molecule) glycoprotein is a transmembrane glycoprotein that serves as a co-receptor for the T cell receptor (TCR). CD8 binds to a major histocompatibility complex (MHC) molecule, but is specific for the class I MHC protein. Exemplary CD8 T cells comprise cytotoxic memory CD8 T cells, regulatory CD8 T cells, cytotoxic effector CD8 T-cells and additional cells identifiable by a skilled person.

The terms “CMV” or “Cytomegalovirus”, as used herein, refers to a genus of viruses in the order Herpesvirales, family Herpesviridae, subfamily Betaherpesvirinae. The CMV genus comprises 11 species including human betaherpesvirus 5 (HCMV, human cytomegalovirus, HHV-5), which is the species that infects humans. Diseases associated with HHV-5 include mononucleosis and pneumonia (ENA accession number GU980198).

The term “comprising” or “comprises”, as used herein, discloses also “consisting of” according to the generally accepted patent practice.

The term “controller”, as used herein, refers to individuals who have been infected with HIV and that exhibit either (i) undetectable plasma HIV RNA or (ii) detectable but low levels of plasma HIV RNA (Deeks S, et al., Immunity 2007; 27:406-416, Ferre A, et al., Blood 2009; 113(17):3978-3989).

The terms “Coronavirus”, as used herein, refers to any member of the Coronaviridae viral family. The Coronaviridae family includes single-stranded RNA viruses, about 120 nanometers in diameter. The family is divided in two subfamilies: Letovirinae and Coronavirinae. The Coronavirinae subfamily comprises the Alphacoronavirus (e.g., human coronavirus 229E (HCoV-229E), Betacoronavirus (e.g., human coronavirus HKU1, human coronavirus NL63 (HCoV-NL63, New Haven coronavirus), human coronavirus OC43 (HCoV-OC43), Middle East respiratory syndrome-related coronavirus (MERS-CoV or HCoV-EMC, the causative agent of MERS), severe acute respiratory syndrome coronavirus (SARS-CoV, the causative agent of SARS), severe acute respiratory syndrome coronavirus 2 (SARS-CoV-2, the causative agent of COVID-19), Deltacoronavirus, and Gammacoronavirus genus. Coronaviruses can also infect non-human subjects such as, for example, cattle (e.g., bovine coronavirus (BCV), cats (e.g., feline coronavirus (FCoV), dogs (e.g., canine coronavirus (CCoV), pigs (e.g., porcine coronavirus HKU15, porcine epidemic diarrhea virus (PED or PEDV), rabbits (e.g., rabbit enteric coronavirus), and birds (e.g., infectious bronchitis virus (IBV), turkey coronavirus (TCV)). There are more than 40 species of Coronaviruses.

The term “cytokine”, as used herein, refers to small proteins secreted by cells that can alter the behavior or properties of the secreting cell or another cell. Cytokines bind to cytokine receptors and trigger a behavior or property within the cell, for example, cell proliferation, death or differentiation. Exemplary cytokines include, but are not limited to, interleukins (e.g., IL-2, IL-3, IL-4, IL-5, IL-6, IL-7, IL-8, IL-9, IL-10, IL-11, IL-12, IL-13, IL-15, IL-16, IL-17, IL-18, IL-la, IL-1 beta, and IL-1 RA), granulocyte colony stimulating factor (G-CSF), granulocyte-macrophage colony stimulating factor (GM-CSF), oncostatin M, erythropoietin (EPO), leukemia inhibitory factor (LIF), interferons, TNF family members (e.g., TNF-α, TNF-β, LT-β). The cytokine levels and cells producing them can be determined by several methods well-known in the art, such as ELISA, ELISPOT, Western Blot, and flow cytometry, with different levels of sensitivity and specificity.

The term “chemokine”, as used herein, refers to chemotactic cytokines secreted by cells that induce directional movement of leukocytes, as well as other cell types. Exemplary chemokines are CC chemokines (CCL1-CCL28), CXC chemokines (CXCL1-CXCL17), C chemokines (XCL1 and XCL2) and CX3C chemokines, such as CX3CL1. In the present invention, chemokine CXCL13 is of special relevance.

The term “EBV” or “Epstein-Barr virus”, as used herein, refers to a double-stranded DNA virus member of the herpes virus family (Zanella M, et al., Clinical Microbiol Rev 2020; 33(4):e00027-20.0). EBV spreads most commonly through bodily fluids, primarily saliva. EBV can cause infectious mononucleosis, also called mono or kissing disease virus, and other illnesses, in particular several forms of cancer such as lymphomas and carcinomas.

The term “ELISPOT” or “enzyme-linked immunospot”, as used herein, refers to a highly sensitive immunoassay that measures the frequency of cytokine-secreting cells at the single-cell level. In this assay, cells are cultured on a surface coated with a cytokine-specific capture antibody in the presence or absence of antigenic stimuli. Either a monoclonal or polyclonal antibody specific for the chosen analyte (e.g., cytokine) is usually pre-coated onto a PVDF (polyvinylidene difluoride)-backed microplate. Appropriately stimulated cells are pipetted into the wells and the microplate is placed into a humidified 37° C. CO₂ incubator for a specified period of time. During this incubation period, the immobilized antibody, in the immediate vicinity of cells secreting the analyte of interest, binds to the secreted analyte. After washing away any cells and unbound substances, a biotinylated polyclonal antibody specific for the chosen analyte is added to the wells. Following a wash to remove any unbound biotinylated antibody, streptavidin conjugated with an enzyme (e.g., alkaline phosphatase) is added. Unbound enzyme is subsequently removed by washing and an enzyme specific substrate solution (e.g., BCIP/NBT) is added. A colored precipitate (e.g., blue-black in the case of BCIP/NBT) forms and appears as spots at the sites of cytokine localization, with each individual spot representing an individual analyte-secreting cell. Biotin-streptavidin enzymatic detection can be replaced for fluorescent labelling allowing for direct detection of the secreted analyte by a fluorescence detecting apparatus. The spots can be counted with an automated ELISPOT reader system or manually, using a stereomicroscope.

The term “HBV” or “Hepatitis B virus”, as used herein, refers to a double-stranded DNA virus, a species of the genus Orthohepadnavirus and a member of the Hepadnaviridae family of viruses. HBV is associated with acute and chronic hepatitis and may lead to the development of liver cirrhosis and hepatocellular carcinoma (Ryu W, Molecular Virology of Human Pathogenic Viruses (Academic Press, Cambridge, MA, USA, 2017, p. 247-260)).

The term “HCV” or “Hepatitis C virus”, as used herein, refers to a single-stranded, positive-sense RNA virus member of the genus Hepacivirus in the family Flaviviridae (Rosen H, et al., NEJM 2011; 364(25):2429-2438). HCV may lead to liver disease and cirrhosis. In some cases, those with cirrhosis may develop serious complications such as liver failure, liver cancer, or dilated blood vessels in the esophagus and stomach.

The term “HIV”, as used herein, include HIV-1, HIV-2 and SHIV. “HIV-1” means the human immunodeficiency virus type-1. HIV-1 includes, but is not limited to, extracellular virus particles and the forms of HIV-1 associated with HIV-1 infected cells. The HIV-1 virus may represent any of the known major subtypes (Classes A, B, C, D E, F, G and H) or outlying subtype (Group O) including laboratory strains and primary isolates. “HIV-2” means the human immunodeficiency virus type-2. HIV-2 includes, but is not limited to, extracellular virus particles and the forms of HIV-2 associated with HIV-2 infected cells.

The term “SIV” refers to simian immunodeficiency virus which is a primate HIV-homolog virus that infects monkeys, chimpanzees, and other nonhuman primates. SIV includes, but is not limited to, extracellular virus particles and the forms of SIV associated with SIV infected cells.

The term “HIV exposure”, as used herein, refers to the contact of an HIV-uninfected individual with a person having an HIV infection, or the contact with body fluids from such HIV-infected subject, in which such fluids from the infected subject contact a mucous membrane, a cut or abrasion in the tissue (e.g., needle stick, unprotected sexual intercourse), or other surface of the uninfected subject in such a way that the virus could be transmitted from the infected person or infected person's body fluids to the uninfected individual.

The term “HIV infection”, as used herein, refers to indications of the presence of the HIV virus in an individual including asymptomatic seropositivity, AIDS-related complex (ARC), and acquired immunodeficiency syndrome (AIDS).

The term “non-controller”, as used herein, refers to an individual that is infected with HIV and that exhibits disease progression following the initial infection leading to increased viral load, CD4+ T-cell loss and consequent and immune system decay.

The term “kit”, as used herein, refers to a product containing the different reagents necessary for carrying out the method of the invention which is packed so as to allow their transport and storage. Materials suitable for packing the components of the kit include crystal, plastic (e.g., polyethylene, polypropylene, polycarbonate), bottles, vials, paper and/or envelopes.

The term “M. tuberculosis”, as used herein, refers a complex which comprises at least nine species (i.e., M. tuberculosis sensu stricto, M. africanum, M. canetti, M. bovis, M. caprae, M. microti, M. pinnipedii, M. mungi, M. orygis) in the genus Mycobacterium, family Mycobacteriaceae, and order Actinomycetales that are causes of human tuberculosis and zoonotic disease.

The term “pathogen”, as used herein, refers to a biological agent that causes a disease state (e.g., infection, cancer) in a host. “Pathogens” include, but are not limited to, viruses, bacteria, archaea, fungi, protozoans, mycoplasma, prions, and parasitic organisms.

The term “PBMC”, as used herein, refers to peripheral blood mononuclear cells, including lymphocytes, monocytes and macrophages. PBMCs may be isolated using methods known in the art, such as density gradient centrifugation.

The term “subject”, as used herein, refers to an individual or animal, such as a human, a nonhuman primate (e.g., chimpanzees and other apes and monkey species); farm animals, such as birds, fish, cattle, sheep, pigs, goats and horses; domestic mammals, such as dogs and cats; laboratory animals including rodents, such as mice, rats and guinea pigs. The term does not denote a particular age or sex. The term “subject” encompasses an embryo and a fetus. In some embodiments, the subject is a human.

The term “T cell response”, as used herein, refers to an immune response in which T cells directly or indirectly mediate or otherwise contribute to an immune response in a mammal. The T cell mediated immune response may be associated with cell mediated effects (e.g., lymphokine mediated effects), and even effects associated with B cells if the B cells are stimulated, for example, by the lymphokines secreted by T cells. For MHC class I restricted CTLs, effector functions may be lysis of peptide-pulsed, peptide-precursor pulsed or naturally peptide-presenting target cells, secretion of cytokines (e.g., IFN-γ, TNF-α, IL-2), secretion of effector molecules (e.g., granzymes, perforins), or degranulation. For MHC class Il-restricted T helper cells, effector functions may be peptide induced secretion of cytokines (e.g., IFN-γ, TNF-α, IL-4, IL5, IL-10, IL-13, IL-2), or degranulation but in some cases can also include other effector functions, such s cytotoxic activity. T cell responses can also be measured as proliferation of T cells and can be observed in vivo as well as in vitro.

### 2. Method of Assaying T Cell Responses

In a first aspect, the present invention relates to a method for assaying polarized T cell responses specific against an antigen in a subject which comprises:
(iii) contacting a biological sample comprising T cells from the subject with a composition comprising the antigen and
(iv) determining the levels of a plurality of cytokines and/or chemokines produced by the T cells in the sample by boosted flow cytometry and/or boosted ELISPOT.
wherein a combination of antibodies against the plurality of cytokines and/or chemokines is used and the plurality of cytokines and/or chemokines is related to a specific T-cell polarization profile,
wherein the antigen is at least one antigen from M. tuberculosis.

In a preferred embodiment, the antigen comprises at least one M. tb protein, preferably ESAT-6, CFP-10, or a combination thereof.

In a preferred embodiment, the plurality of cytokines and/or chemokines comprises cytokines and/or chemokines related to a Type 1 (Th1/Tc1), Type 2 (Th2/Tc2), Type 3 (Th17/Tc17), regulatory T-cell (Treg), and/or follicular T-cell (Tfh/Tfc) polarization profile, and/or other minor emergent polarization profiles, such as Th9 and Th22. Preferably, the plurality of cytokines and/or chemokines comprises cytokines and/or chemokines related to a Type 1 (Th1/Tc1) and/or a Type 2 (Th2/Tc2) polarization profile.

In a preferred embodiment, the Type 1 (Th1/Tc1) plurality of cytokines comprises IFN-7, IL-2, TNF-α, or a combination thereof. In a preferred embodiment, the Type 2 (Th2/Tc2) plurality of cytokines comprises IL-4, IL-5, IL-10, IL-13, or a combination thereof. In a preferred embodiment, the Type 3 (Th17/Tc17) plurality of cytokines comprises IL-17, IL-22, or a combination thereof. In a preferred embodiment, the regulatory T-cell (Treg) plurality of cytokines comprises IL-10 and TGF-β, or a combination thereof. In a preferred embodiment, the follicular T-cell (Tfh/Tfc) plurality of cytokines comprises IL-4, IL-21, CXCL-13 or a combination thereof. In a preferred embodiment, the antibodies comprise monoclonal antibodies. In a preferred embodiment, the sample is a blood sample, preferably a peripheral blood sample. Preferably, the sample to be analyzed is a peripheral blood cells preparation.

In one aspect, the present invention refers to a method for assaying the polarized T cell responses specific against an antigen in a subject which comprises:
(i) contacting a biological sample comprising T cells from the subject with a composition comprising the antigen and
(ii) determining the levels of a plurality of cytokines produced by the T cells in the sample by boosted flow cytometry and/or boosted ELISPOT,
wherein a combination of antibodies against the plurality of cytokines is used and the plurality of cytokines is related to a specific T-cell polarization profile.

In some embodiments, the antibodies are monoclonal antibodies. In some embodiments, the sample containing T cells is a PBMC preparation.

In some embodiments, the antigen comprises a pathogen-derived antigen, a self-antigen, an allergen, a toxin, or a combination thereof. In some embodiments, the antigen comprises a natural or synthetic antigen, or a combination thereof. In some embodiments, the pathogen comprises a HIV, HBV, HCV, Coronavirus, CMV, EBV, M. tuberculosis antigen, or a combination thereof. In some embodiments, the self-antigen comprises a chronic inflammatory demyelinating polyneuropathy, Graves' disease, Guillain-Barré syndrome, Hashimoto's thyroiditis, inflammatory bowel disease (IBD), multiple sclerosis (MS), myasthenia gravis, psoriasis, rheumatoid arthritis (RA), systemic lupus erythematosus, type 1 diabetes mellitus, vasculitis antigen, or a combination thereof.

In some embodiments, the plurality of cytokines comprises cytokines related to a Type 1 (Th1/Tc1), Type 2 (Th2/Tc2), Type 3 (Th17/Tc17), regulatory T-cell (Treg), or follicular T-cell (Tfh/Tfc) polarization profile. In some embodiments, the Type 1 (Th1/Tc1) plurality of cytokines comprises IFN-γ, IL-2, TNF-α, or a combination thereof. In some embodiments, the Type 2 (Th2/Tc2) plurality of cytokines comprises IL-4, IL-5, IL-10, IL-13, or a combination thereof. In some embodiments, the Type 3 (Th17/Tc17) plurality of cytokines comprises IL-17, IL-22, or a combination thereof. In some embodiments, the regulatory T-cell (Treg) plurality of cytokines comprises IL-10 and TGF-β, or a combination thereof. In some embodiments, the follicular T-cell (Tfh/Tfc) plurality of cytokines comprises IL-4, IL-21, CXCL-13 or a combination thereof.

In some embodiments, the plurality of cytokines comprises cytokines related to rarer subsets of T-cell polarization profiles (e.g., Th22, Th9, others).

In some embodiments, the method of the invention could be useful for analyzing the polarization of cellular immunity in different immune-settings, including infectious diseases (e.g., HIV, M. tuberculosis, SARS-Cov2), autoimmune diseases (e.g., MS, RA), allergic and toxic reactions. In some embodiments, the method of the invention could be used for monitoring the treatment of a subject for the underlying disease, such as HIV infection or infections by other pathogens, cancer, and/or autoimmune diseases.

### 3. Kits

In a second aspect, the present invention relates to a kit comprising reagents for the detection of cytokines and/or chemokines according to the method of the first aspect. In a preferred embodiment, the kit comprises reagents for detecting one or more cytokines related to a Type 1 (Th1/Tc1), Type 2 (Th2/Tc2), Type 3 (Th17/Tc17), regulatory T-cell (Treg), Th22, Th9, or follicular T-cell (Tfh) polarization profile. In some embodiments, the kit comprises antibodies for the detection of one or more cytokines selected from the groups consisting of IFN-γ, IL-2, IL-4, IL-5, IL-9, IL-10, IL-13, IL-17, IL-21, IL-22, TGF-β, CXCL-13 and TNF-α.

In a further aspect, the present invention refers to kits comprising reagents for the detection of one or more cytokines related to a Type 1 (Th1/Tc1), Type 2 (Th2/Tc2), Type 3 (Th17/Tc17), regulatory T-cell (Treg), Th22, Th9, or follicular T-cell (Tfh) polarization profile. In some embodiments, the kit comprises antibodies for the detection of one or more cytokines selected from the groups consisting of IFN-γ, IL-2, IL-4, IL-5, IL-9, IL-10, IL-13, IL-17, IL-21, IL-22, TGF-β, CXCL-13 and TNF-α. The components of the kits of the invention may be optionally packed in suitable containers and be labeled for detecting the antigens of the invention (e.g, pathogen-derived antigens such as HIV, HBV, HCV, Coronavirus, CMV, EBV, and M. Tuberculosis antigens; self-antigens such as chronic inflammatory demyelinating polyneuropathy, Graves' disease, Guillain-Barré syndrome, Hashimoto's thyroiditis, inflammatory bowel disease (IBD), multiple sclerosis (MS), myasthenia gravis, psoriasis, rheumatoid arthritis (RA), systemic lupus erythematosus, type 1 diabetes mellitus, and vasculitis antigens) and cancer antigens (e.g., p53-derived T cell antigens). The components of the kits may be stored in unit or multi-dose containers as an aqueous, preferably sterile, solution or as a lyophilized, preferably sterile, formulation for reconstitution. The containers may be formed from a variety of materials such as glass or plastic and may have a sterile access port (e.g., the container may be an intravenous solution bag or a vial having a stopper pierceable by a hypodermic injection needle). The kits may further comprise more containers comprising a pharmaceutically acceptable carrier. They may further include other materials desirable from a commercial and user standpoint, including, but not limited to, buffers, diluents, filters, needles, syringes, or other active agents. The kits can contain instructions customarily included in commercial packages of diagnostic and therapeutic products that contain information, for example, about the indications, usage, dosage, manufacture, expiration, administration, contraindications or warnings concerning the use of such diagnostic products.

### 4. Uses

In a further aspect, the present invention relates to the use of the method of the first aspect or the kit of the second aspect for the diagnosis of tuberculosis. The method and kit of the present invention allows for the diagnosis of active tuberculosis or latent tuberculosis. Importantly, the present invention is of particular relevance for a population selected from children, elderly, immunocompromised patients, subject coinfected with HIV-1, subject under immunosuppressive treatment, and any combination thereof.

When the present invention relates to HIV, preferably HIV-1, the present invention is described as follows: A method for assaying polarized T cell responses specific against an antigen in a subject which comprises:
(i) contacting a biological sample comprising T cells from a subject with a composition comprising the antigen and
(ii) determining the levels of a plurality of cytokines produced by the T cells in the sample by boosted flow cytometry and/or boosted ELISpot,
wherein a combination of antibodies against the plurality of cytokines is used and the plurality of cytokines is related to a specific T-cell polarization profile, wherein said antigen is at least one antigen from HIV-1. Optionally, the antigen could also comprise a HBV, HCV, Coronavirus, CMV, EBV, M. tuberculosis antigen, or a combination thereof. Where other antigens were present, when the antigen is a self-antigen, said self-antigen may comprise a chronic inflammatory demyelinating polyneuropathy, Graves' disease, Guillain-Barré syndrome, Hashimoto's thyroiditis, inflammatory bowel disease (IBD), multiple sclerosis (MS), myasthenia gravis, psoriasis, rheumatoid arthritis (RA), systemic lupus erythematosus, type 1 diabetes mellitus, vasculitis antigen, or a combination thereof.

In a preferred embodiment of the method, the plurality of cytokines comprises cytokines related to a Type 1 (Th1/Tc1), Type 2 (Th2/Tc2), Type 3 (Th17/Tc17), regulatory T-cell (Treg), or follicular T-cell (Tfh/Tfc) polarization profile or other, minor emergent polarization profiles. Preferably, the Type 1 (Th1/Tc1) plurality of cytokines comprises IFN-γ, IL-2, TNF-α, or a combination thereof. Preferably, the Type 2 (Th2/Tc2) plurality of cytokines comprises IL-4, IL-5, IL-10, IL-13, or a combination thereof. Preferably, the Type 3 (Th17/Tc17) plurality of cytokines comprises IL-17, IL-22, or a combination thereof. Preferably, the regulatory T-cell (Treg) plurality of cytokines comprises IL-10 and TGF-0, or a combination thereof. Preferably, the follicular T-cell (Tfh/Tfc) plurality of cytokines comprises IL-4, IL-21, CXCL-13 or a combination thereof. In a preferred embodiment, the antibodies comprise monoclonal antibodies. Preferably, the sample to be analyzed is a peripheral blood sample, optionally a peripheral blood cells preparation. Another aspect is a kit comprising reagents for the detection of cytokines as described above. Also, another aspect is the use of the method or kit for the diagnosis of HIV diagnosis and immune monitoring.

All publications mentioned herein are incorporated in their entirety by reference. Having now generally described the invention, the same will be more readily understood through reference to the following examples, which are provided by way of illustration and are not intended to be limiting of the present invention, unless specified.

### General Procedures

### 1. Study Design

(a) TB: The objective of the study was identifying M. Tb-specific T cell responses of alternative (non-unique IFN-γ-mediated) effector function profiles predictive of M. Tb infection and the discriminating ability between Active and Latent infection disease (ATBI and LTBI), in M.Tb. infected individuals. Twenty-two M. Tb infected individuals were included in the study. Ten participants had an Active TB infection (ATBI) diagnosis based on microbiology culture determination and pulmonary X-Ray and complemented by an additional positive test measured by Bacilloscopy, PCR and/or TST, QFN, TB T-SPOT (see Table 1). Twelve participants had a Latent TB infection (LTBI) diagnosis based on negative micobacteria location, X-Ray, undetermined/negative microbiology culture, Bacilloscopy and PCR, but positive for TST, QFN and TB T-SPOT.

(b) HIV: One of the initial objectives of the described exemplary study was identifying HIV-specific T cell responses of alternative (non-IFN-γ-mediated) effector function profiles that are associated with critical aspects of the humoral immune responses to HIV. Forty-four HIV-infected, treatment naïve individuals (HIV+), and 8 HIV seronegative controls (HIV−) were included in the study. Twenty-four participants had viral loads (VL)<2000 copies/mL and were considered HIV controllers. They showed a median VL of 210 copies/mL (VL range: 25-1900 copies/mL) and a median CD4+ T cell count of 786 cells/mm³ (CD4+ T cell count range: 364-1638 cells/mm³). An additional 20 participants presented with VL>2000 copies/mL (median 52435 copies/mL, range 5576-640803) and were considered non-controllers (median CD4+ T-cell count: 406 cells/mm³, range: 6-986 cells/mm³). Plasma samples from all the individuals were tested for anti-Env IgM, IgA and IgG titers as well as the plasma neutralization titters and ADCC activity. Due to sample availability, boosted flow analysis for cellular responses included 29 individuals (15 HIV controllers and 14 HIV non-controllers) and phenotypic characterization based on Th2/Tc2 markers (CRTH2) and Tfh/Tfc markers (CXCR5+ PD-1+ ICOS+) was performed in twenty-two of the samples used in the boosted flow analysis (8 HIV controllers and 14 HIV non-controllers). The same 22 individuals were also tested by the boosted ELISPOT method with the same antigens as used in the boosted flow cytometry tests. Ex-vivo experiments were performed without blinding or randomization.

### 2. Boosted Flow Screening of TB or HIV-Specific T-Cell Responses

(a) TB: Cryopreserved peripheral mononuclear cells (PBMC) from 10 ATBI and 12 LTBI individuals were thawed and stained. 5×10⁵ PBMC/well were cultured in R10 (RPMI medium (Gibco) supplemented with 2 mM L-glutamine (Gibco), 100 U/mL penicillin (Gibco), 100 μg/mL streptomycin (Gibco) and 10% heat-inactivated inactivated fetal bovine serum (FBS, Invitrogen), for 16 h at 37° C. PBMCs were stimulated with ESAT-6, CFP-10, ESAT6+CFP-10 proteins and PPD protein as positive control, in the presence of anti-CD49d and anti-CD28 (BD Biosciences) and GolgiStop (BD Biosciences). As negative controls, culture medium was used instead of peptide pools. Cells were stained first with BD Horizon™ Fixable Viability Stain 575V, followed by extracellular staining for T-cell lineage markers (anti-CD3 APC-Cy7, anti-CD4 BV786, anti CD8-PerCP, anti-CCR7 BV711, Biolegend; anti-CD45RA FITC, BD Biosciences), B-cell lineage marker (anti-CD19 PE-Dazzle594, Biolegend) and myeloid lineage marker (anti-CD14 A700, Biolegend). Following the fixation and permeabilization step (Fix and Perm kit, Invitrogen) intracellular staining (ICS) was performed, following a boosting strategy described in the art (Ruíz-Riol M, et al., J. Infect. Dis. 2015; doi:10.1093/infdis/jiu534; Romero-Martin, L et al. Front Immunol. 2022; 13: 928039) for Type 1, Type 2, Type 3, Treg and T follicular defining cytokines. Antibodies against Type 1 related cytokines were conjugated with BV605 (anti-IFN-α, anti-IL-2, anti-TNF-α, Biolegend), antibodies against Type 2 related cytokines with BV421 (anti-IL-4, anti-IL-10, anti-IL-13, Biolegend), antibodies against Type 3 related cytokines with PE-Cy7 (anti-IL-17-A, anti-IL-22, Biolegend), antibodies against Treg related cytokines with PE (anti-IL-10, anti-LAP, Biolegend) and antibodies against T follicular related cytokines with APC (anti-IL-4, anti-IL-21, Biolegend). Cells were classified as TCM (CD45RA− CCR7+), TEM (CD45RA− CCR7−), TEMRA (CD45RA+ CCR7−) and TNaive (CD45RA+ CCR7+).

Approximately 10⁵ cells were acquired on a LSR Fortessa BD Instrument and analysis was performed using FlowJo 10.5.2. See FIG. 1). The percentage of cytokine-producing cells (i.e., magnitude) detected in unstimulated controls was subtracted from the magnitude of the antigen-stimulated cells. The value of the most negative magnitude value after control subtraction across all individuals determined the threshold for positive responses, as previously described (Roederer M, et al., Cytom. Part A 2011; doi:10.1002/cyto.a.21015). Polyfunctional cytokine profiling was done applying Boolean gates in FlowJo 10.5.2, following the same strategy for unstimulated cell signal subtraction and represented using SPICE v6 (US National Institutes of Health, ImmunoTecnhnology Section, Vaccine Research Center, NIAID, NIH, Bethesda, MD, USA).

(b) HIV: Cryopreserved peripheral mononuclear cells (PBMC) from 15 HIV+ controllers and 14 HIV+ non-controllers were thawed and stained. 5×10⁵ PBMC/well were cultured in R10 (RPMI medium (Gibco) supplemented with 2 mM L-glutamine (Gibco), 100 U/mL penicillin (Gibco), 100 μg/mL streptomycin (Gibco) and 10% heat-inactivated inactivated fetal bovine serum (FBS, Invitrogen), for 16 h at 37° C. PBMCs were stimulated with a set of 425 18mer overlapping peptides (OLPs) divided in 17 peptide pools (range 21-27 OLPs/pool, final concentration for each peptide 10 μg/mL) covering all the HIV proteome or anti-CD3/anti-CD28 magnetic beads (Gibco) as positive control, in the presence of anti-CD49d and anti-CD28 (BD Biosciences) and GolgiStop (BD Biosciences). As negative controls, culture medium was used instead of peptide pools. Cells were stained first with BD Horizon™ Fixable Viability Stain 575V, followed by extracellular staining for T-cell lineage markers (anti-CD3 APC-Cy7, anti-CD4 BV786, anti CD8-PerCP, anti-CCR7 BV711, Biolegend; anti-CD45RA FITC, BD Biosciences), B-cell lineage marker (anti-CD19 PE-Dazzle594, Biolegend) and myeloid lineage marker (anti-CD14 A700, Biolegend). Following the fixation and permeabilization step (Fix and Perm kit, Invitrogen) intracellular staining (ICS) was performed, following a boosting strategy described in the art (Ruíz-Riol M, et al., J. Infect. Dis. 2015; doi:10.1093/infdis/jiu534; Romero-Martin L, et al. Front Immunol. 2022; 13: 928039) for Type 1, Type 2, Type 3, Treg and T follicular defining cytokines. Antibodies against Type 1 related cytokines were conjugated with BV605 (anti-IFN-α, anti-IL-2, anti-TNF-α, Biolegend), antibodies against Type 2 related cytokines with BV421 (anti-IL-4, anti-IL-10, anti-IL-13, Biolegend), antibodies against Type 3 related cytokines with PE-Cy7 (anti-IL-17-A, anti-IL-22, Biolegend), antibodies against Treg related cytokines with PE (anti-IL-10, anti-LAP, Biolegend) and antibodies against T follicular related cytokines with APC (anti-IL-4, anti-IL-21, Biolegend). Cells were classified as TCM (CD45RA− CCR7+), TEM (CD45RA− CCR7−), TEMRA (CD45RA+ CCR7−) and TNaive (CD45RA+ CCR7+).

Approximately 10⁵ cells were acquired on a LSR Fortessa BD Instrument and analysis was performed using FlowJo 10.5.2., FIG. 3). The percentage of cytokine producing cells (i.e., magnitude) detected in unstimulated controls was subtracted from the magnitude of the antigen-stimulated cells. The value of the most negative magnitude value after control subtraction across all individuals determined the threshold for positive responses, as previously described (Roederer M, et al., Cytom. Part A 2011; doi:10.1002/cyto.a.21015). Polyfunctional cytokine profiling was done applying Boolean gates in FlowJo 10.5.2, following the same strategy for unstimulated cell signal subtraction and represented using SPICE v6 (US National Institutes of Health, ImmunoTecnhnology Section, Vaccine Research Center, NIAID, NIH, Bethesda, MD, USA).

### 3. Phenotypic Characterization of Antigen-Specific CD4+ Th2− and Tfh Cells and CD8+ Tc2− and Tfc− Cells

Surface markers for Th2/Tc2 (CRTH2) and Tfh/Tfc T cell polarization (CXCR5, ICOS and PD-1) were characterized in cryopreserved PBMC from 8 HIV+ controllers and 14 HIV+ non-controllers, previously yielding T cell responses in the boosted flow whole proteome analysis. Cells were thawed and plated at 5×10⁵ PBMCs/well in R10 for 16 h at 37° C. PBMCs were stimulated with peptide pools that showed activation of T cells in the initial screening or control stimulations as above. Cells were stained first with BD Horizon™ Fixable Viability Stain 575V, followed by extracellular staining for T-cell lineage markers (anti-CD3 APC-Cy7, anti-CD4 BV786, anti-CD8-PerCP, anti-CCR7 BV711, Biolegend; anti-CD45RA FITC, BD Biosciences), Th2/Tc2 marker (anti-CRTH2 BV605, Biolegend) and Tfh/Tfc markers (anti-CXCR5 PE, anti-ICOS PE-Dazzle594, anti-PD-1 PE-Cy7, Biolegend). Following the fixation and permeabilization step (Fix and Perm kit, Invitrogen) intracellular staining was performed using anti-IL-4 BV421 and anti-IL-21 APC (Biolegend).

Approximately 10⁵ cells were acquired on a LSR Fortessa BD Instrument and analysis was performed using FlowJo 10.5.2 software. Additionally, data sets from CD3+ cells of the negative control phenotypic characterization were down sampled to obtain 3000 events/fcs file. An unsupervised UMAP dimensional reduction was performed on FlowJo 10.5.2 using the default settings of a nearest neighbors of 15 and a minimum Euclidean distance of 0.5. The number of clusters was determined by X-Shift (Number nearest neighbors: 132, Euclidean distance metric) and FlowSOM and was applied to determine the level of expression of our proteins of interest in a total of 45 clusters in the CD3+ population. The abundance of each cluster was determined in HIV+ controllers and non-controllers and results were represented for each cluster as log 2(% CD3+ cells in C/% CD3+ cells in NC).

### 4. IL-4, IL-21 and IFN-γ Boosted ELISPOT

Mabtech ELISPOT kits were used to count IFN-7 (capture clone 1-D1K, biotinylated clone 7-B6-1), IL-4 (capture clone IL-4I, biotinylated clone IL-4II) and IL-21 (capture clone MT216G, biotinylated clone MT21.3m) producing cells following manufacturer's instructions. An additional assay, analogous to the boosted Flow technology, was performed to enhance the sensitivity of detection by ELISPOT. To that end, plates were coated with multiple capturing antibodies to allow for the detection of IL-4 and IL-21-producing cells. A total of 2×10⁵ PBMCs/well from 8 HIV+ controllers and 14 HIV+ non-controllers were stimulated for 48 h at 37° C. with peptide pools to which the individuals responded in the initial screening, using R10 as negative control in triplicates and PHA (25 μg/mL) as positive control. Spot-forming cells (SFC) were counted using an automated Cellular Technology Limited (C.T.L.) ELISPOT Reader Unit. The threshold for positivity was set as the highest of: a) 50 SFC/10⁶ PBMCs, b) mean SFC in the negative controls plus 3 times standard deviations of the negative control wells or c) 3 times the mean of negative control well SFC/10⁶ PBMCs.

### 5. Determination of Anti-HIV Env IgM, IgA and IgG Titers of Plasma Samples

HIV-1 NL4-3 and HIV-1 BaL infected MOLT cells (Blanco J, et al., J. Leukoc. Biol. 2004; doi:10.1189/jlb.0204100) were pre-incubated at room temperature for 30 minutes with plasma samples diluted 1/300 in PBS 1% BSA (Miltenyi Biotec). After three washes with PBS, the secondary antibodies: PE-F(ab)2 Goat anti-human IgG (Fc-specific); DyLight 649 Goat anti-human IgA and DyLight 488-F(ab)2 Goat anti-human IgM (Fcm5-specific) (Jackson Immunoresearch) were added separately and incubated for 15 minutes at room temperature. After washing, stained cells were washed with PBS two times and fixed in 1% formaldehyde (Sigma-Aldrich). Samples were acquired in a BD LSR II flow cytometer (BD Biosciences) and the analysis was performed using FlowJo 10.5.2 software. HIV-1 NL4-3 and HIV-1 BaL Envelope protein-specific IgM, IgA and IgG titers were correlated to the levels of specifically polarized, virus-antigen specific T cell subsets.

### 6. Neutralization Assays

The neutralizing capacity of plasma from 44 HIV+ individuals was determined using a TZM-bl based pseudovirus neutralization assay as previously described (Molinos-Albert L, et al., Sci. Rep. 2017; 7) using a panel of 5 pseudovirus, including HIV-1 strains classified as tier 1A (NL4-3), 1B (BaL.01), 2 (398F1), TRO.11 and AC10). Briefly, serial dilutions of heat inactivated (56° C. 30 min) plasma samples were incubated with pseudoviruses in D10 (DMEM supplemented with 100 U/mL penicillin (Gibco), 100 μg/mL streptomycin (Gibco) and 10% heat-inactivated FBS (Invitrogen). Afterward, the serial dilutions were added to TZM-bl cells treated with DEAE-Dextran (Sigma), plated at 10⁴ cells/well and cultured at 37° C. for 48 h. Then, 100 μL of the supernatant was replaced with 100 μL of Britelite Luciferase Assay Substrate (Promega) and incubated for 1.5 minutes at room temperature. The cell-associated luciferase signal for each well was determined on an EnSight Multimode Plate Reader (Perkin Elmer). Neutralizing activity for each serum dilution was calculated as the percent of inhibition of viral entry compared to positive and negative controls, untreated infected cells, and uninfected cells, respectively. Neutralization curves (percent viral entry inhibition versus plasma dilution) were generated using GraphPad Prism version 7 by fitting log₁₀-transformed data with a sigmoidal dose-response curve and IC₅₀ was determined. Results were expressed as % of neutralizers in each group, neutralizing breadth (number of HIV-1 strains neutralized/number of HIV-1 strains tested*100) and log IC₅₀ (reciprocal plasma dilution) when data fitted a sigmoidal curve (HIV-1 NL4-3 and HIV-1 BaL).

### 7. Antibody-Dependent Cellular Cytotoxicity (ADCC)

The ability of 44 HIV+ individuals' plasma to induce ADCC was determined by flow cytometry using a modified calcein-AM retention assay previously described (Gillissen M, et al., J. Immunol. Methods 2016; 434). HIV NL4-3-infected NKR-24 target cells were washed and resuspended in 10 nM calcein-AM (BioLegend) diluted in R10 at a cell concentration of 10⁶ cells/mL for 30 minutes with intermittent shakes. Target cells were washed twice with R10, and 10⁴ target cells were co-cultured in R10 supplemented with 10 U/mL rh-IL-2 (Roche) in duplicates for 4 hours with the NK effector cell line N6-ADR at an E:T ratio of 5:1 in the presence or not of serial dilutions (1:1000 to 1:256.000) of HIV+ individuals' plasma. After incubation, cells were washed with PBS plus 1% heat-inactivated FBS (Invitrogen) and 3×10⁴ cells were acquired on a LSRII BD instrument and analyzed on FlowJo 10.5.2 software. Results were normalized with a control condition in the absence of human plasma and lysis was calculated as the average of 100%-% calcein-AM+ cells in each duplicated condition.

### 8. Statistical Analysis

GraphPad Prism version 7 for Mac OS X (La Jolla, CA, USA) was used for statistical analyses. For comparison between group medians, non-parametric Mann-Whitney (MW) test was performed. χ² analysis was used to detect differences in the frequency of T cells with specific secreted cytokine patterns and the number of samples with neutralizing activity. The correlation between the two measures was analyzed using the non-parametric Spearman r test, the correlation p-value was also calculated. Statistical significance criteria were set at p<0.05.

### Example 1: Boosted Flow Screening Identifies a Higher Number of Positive M.Tb-Specific T-Cell Responses with Alternative Cytokine Polarization Compared with Other Gold Standard Assays

An exhaustive screening of cellular responses to M.Tb by boosted flow was performed for evaluating the presence of M.Tb-specific T-cells with alternative (non-IFN-7 dominated, i.e., Th1) cytokine polarization profiles. Total PBMCs from a cohort of 10 ATBI and 12 LTBI were stimulated with different M. tuberculosis proteins commonly tested in other standard assays in the market. Observed signals in the flow cytometry data were expressed as the number of responders (% of individuals eliciting a specific polarized response). As shown in FIG. 1, the Boosted flow ability to capture M. Tb infection was more powerful compared to the other assessments (37% Gold Standard Quantiferon IGRA vs 100% boosted flow in the ATBI and 50% Tuberculin Skin Test vs 100% LTBI).

### Example 2: Boosted Flow Screening Identification of M.Tb-Specific T-Cell Responses with Alternative Cytokine Polarization Associated with ATBI and LTBI

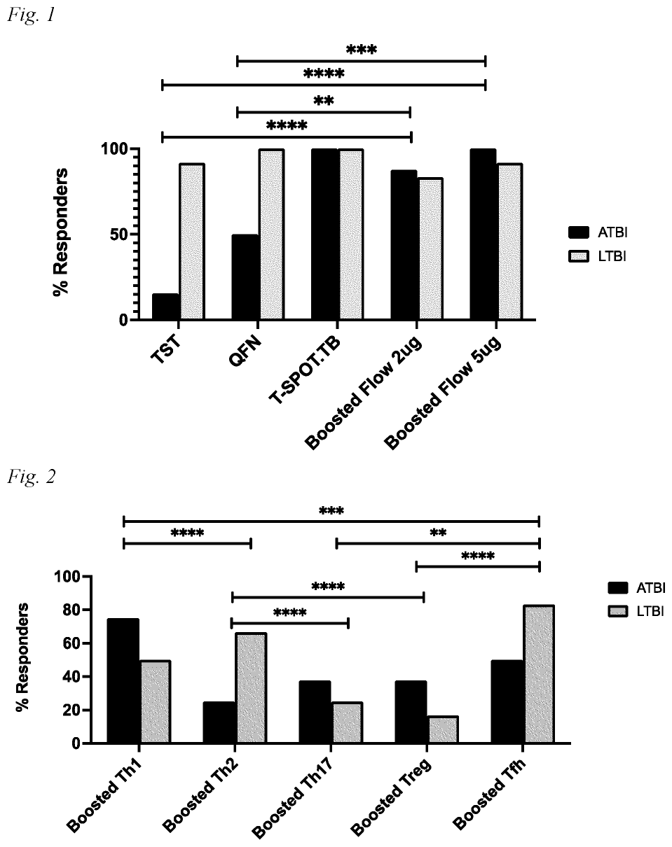

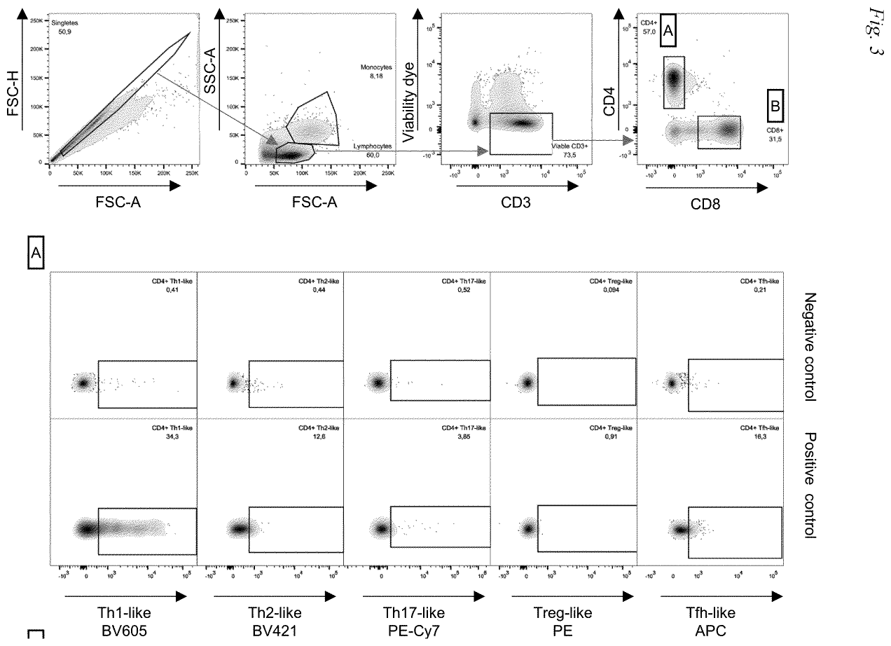

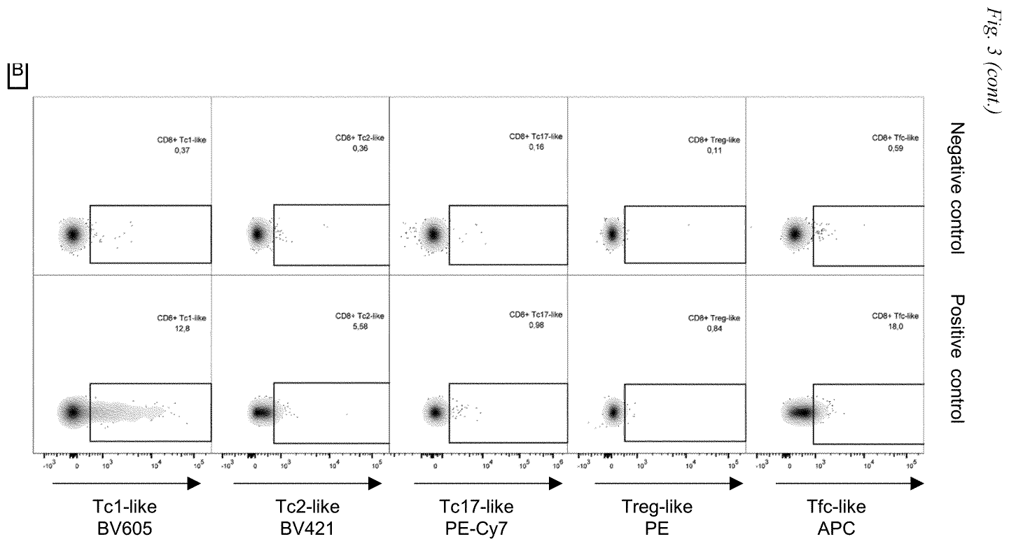

While T-SPOT detects active and latent TB infection at similar rates to Boosted Flow, T-SPOT can not differentiate between the two settings of infection. The breakdown of M. Tb-specific T-cell responses by T-cell polarization profiles allows the discrimination between ATBI and LTBI infection, not possible to achieve with conventional assessments. Moreover, the different polarization profiles are assessed at the same time, in the same sample, without the need for additional parallel tests. As illustrated in FIG. 2, while ATBI predominately generates Th1-like profiles, LTBI generates specific M. Tb responses with Th2-like, Tfh and Th17 effector functions.

### Example 3: Boosted Flow Screening Identification of HIV-Specific T-Cell Responses with Alternative Cytokine Polarization Associated with Viral Control

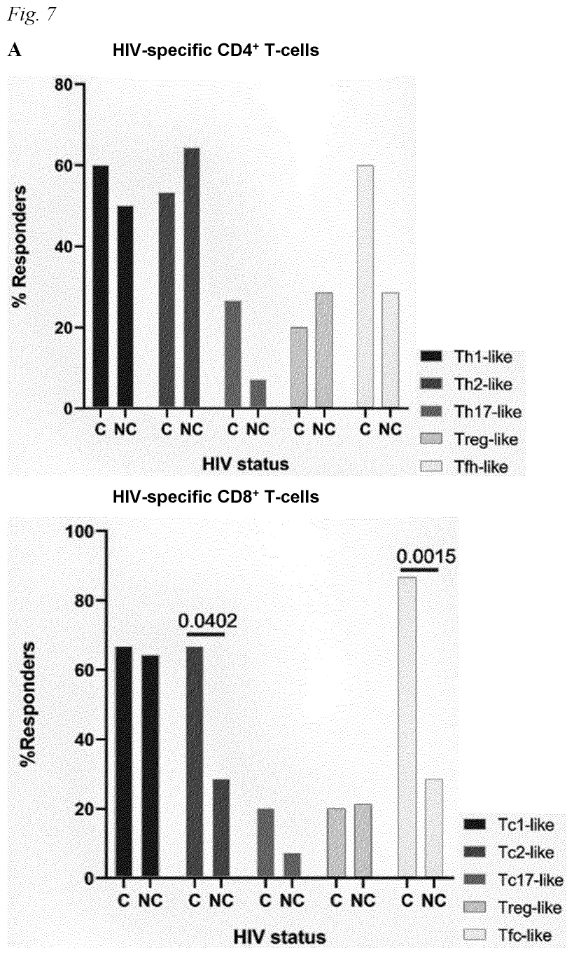

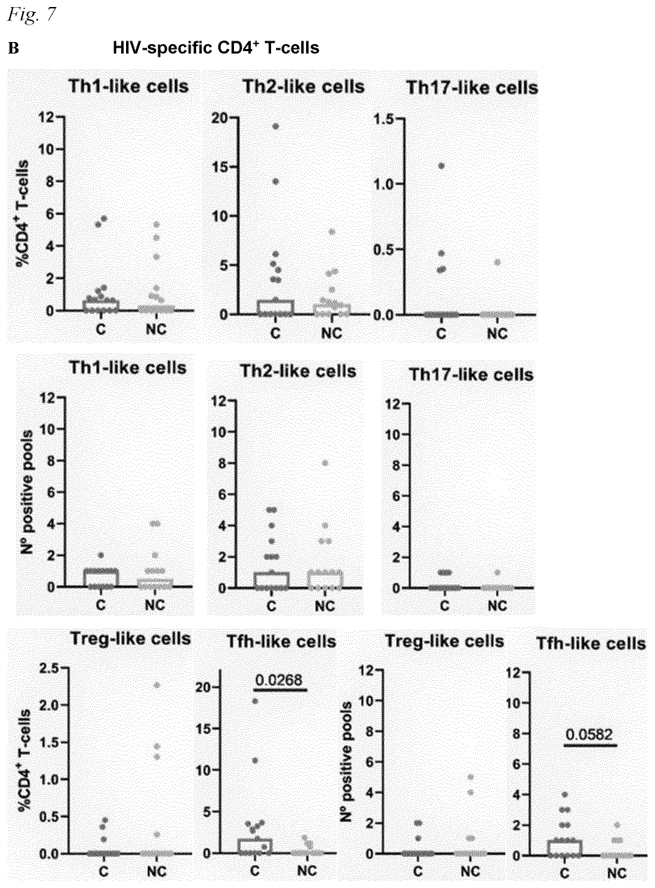

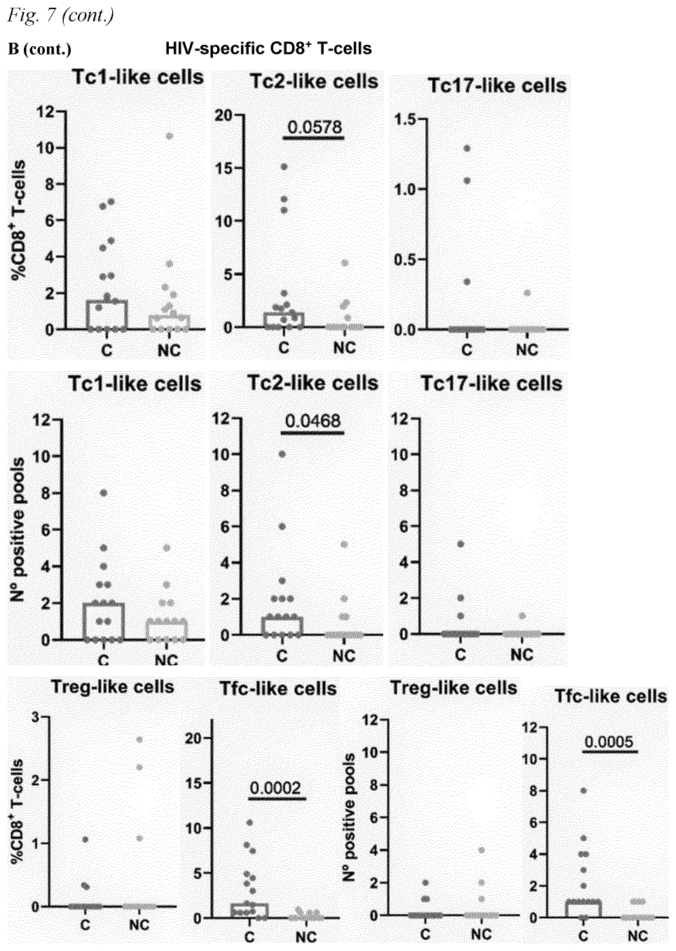

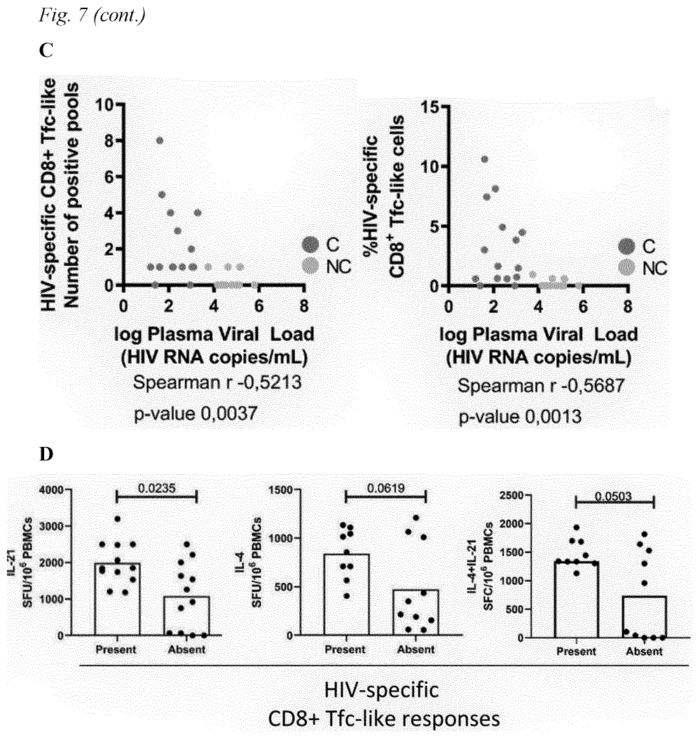

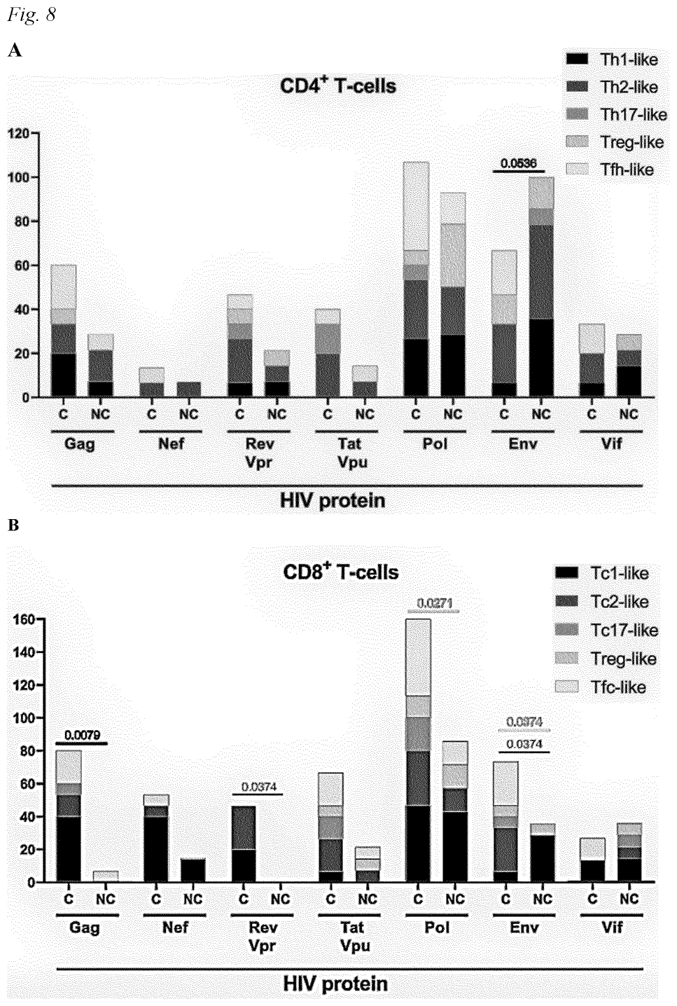

An exhaustive screening of cellular responses to HIV by boosted flow was performed for evaluating the presence of HIV-specific T-cells with alternative (non-IFN-7 dominated, i.e., Th1) cytokine polarization profiles. Total PBMCs from a cohort of 15 HIV+ controllers (C) and 14 HIV+ non-controllers (NC) were stimulated with a set of 17 peptide pools covering the entire HIV proteome. Observed signals in the flow cytometry data were expressed as the number of responders (% of individuals eliciting a specific polarized response), as the breadth (number of reactive peptide pools) and as the magnitude (% of positive cells) of the response. While anti-HIV responses of type-1 CD4+ Th1-like cells and CD8+ Tc1-like cells were the most commonly detected responses, 86% (n=24, including 13 controllers and 11 non-controllers) of the 30 individuals tested also showed non-type-1 like T cell responses. See FIG. 7A. Of these, 5 individuals (2 controllers and 3 non-controllers) elicited exclusively responses with alternative polarization and no type-1 CD4+ or CD8+ T cell responses. The dominant non-type-1 responses were type-2 in CD4+ (percentage of responders: CD4+ Th2-like cells 50%, CD8+ Tc2-like cells 47%) and follicular-like in CD8+ T cells (percentage of responders: CD4+ Tfh-like cells 44%, CD8+ Tfc-like cells 53.5%). Type-3 polarized responses (percentage of responders: CD4+ Th17-like cells 16%, CD8+ Tc17-like cells 13%) and cells with regulatory functions (percentage of responders: CD4+ Treg-like 24%, CD8+ Treg-like 20%) were also detected. See FIG. 7. Together, natural control of HIV infection was associated with increased anti-HIV CD4+ Tfh-like, CD8+ Tc2-like and Tfc-like responses, both response profiles that are not detected by standard intracellular cytokine staining methods.

These findings are further supported by an elevated magnitude (p=0.0268) and breadth (p=0.0582) of HIV-specific CD4+ Tfh-like T-cells (FIG. 7B) in HIV+ controllers compared to non-controllers. CD8+ T-cell mediated responses with Tc2- and Tfc-like profiles were also more frequent (χ² p=0.0402 and 0.0015, respectively) in HIV+ controllers (FIG. 7B), who also showed significantly greater magnitude and breadth of CD8+ Tfc-like responses (magnitude, p=0.0005; breadth p=0.0002) than non-controllers (FIG. 7B). The predominance of these CD8 T cell responses in HIV controllers was further supported by a negative correlation between the breadth and magnitude of HIV-specific CD8+ Tfc-like responses and plasma viral load (FIG. 7C).

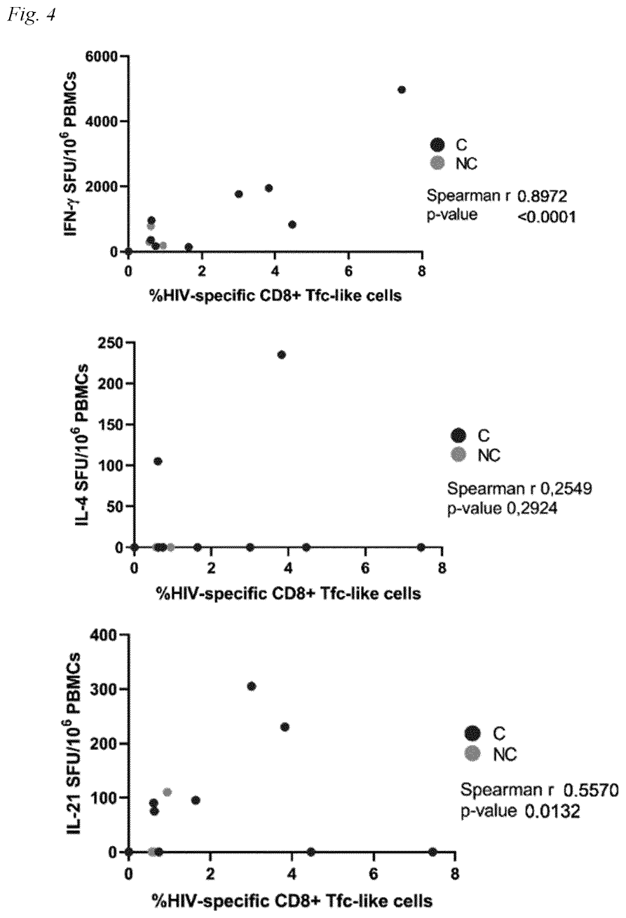

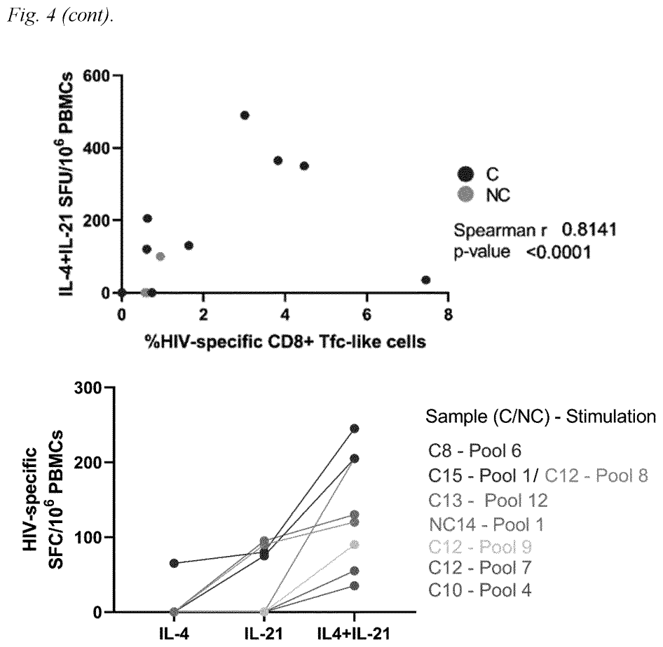

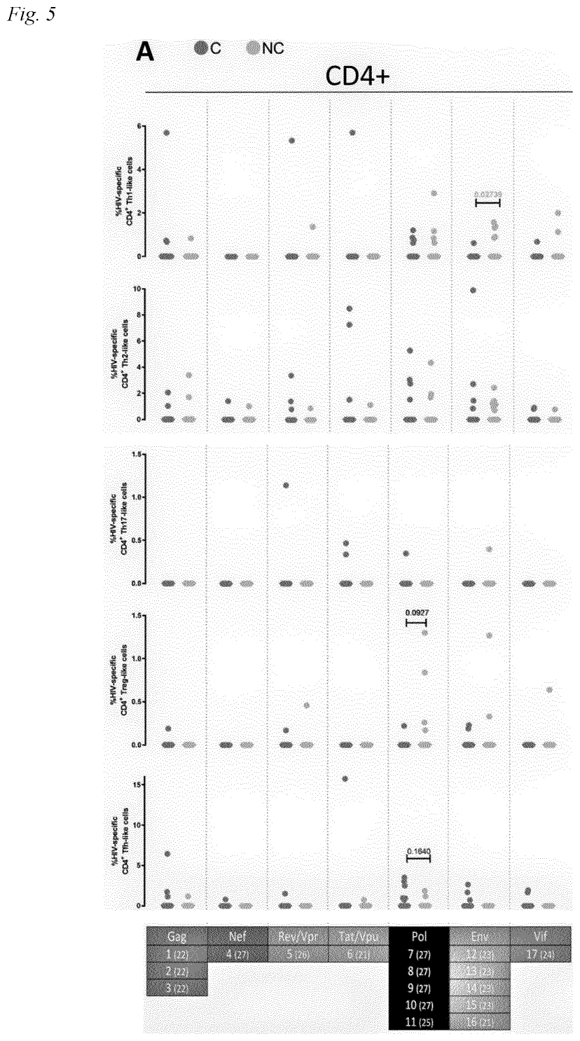

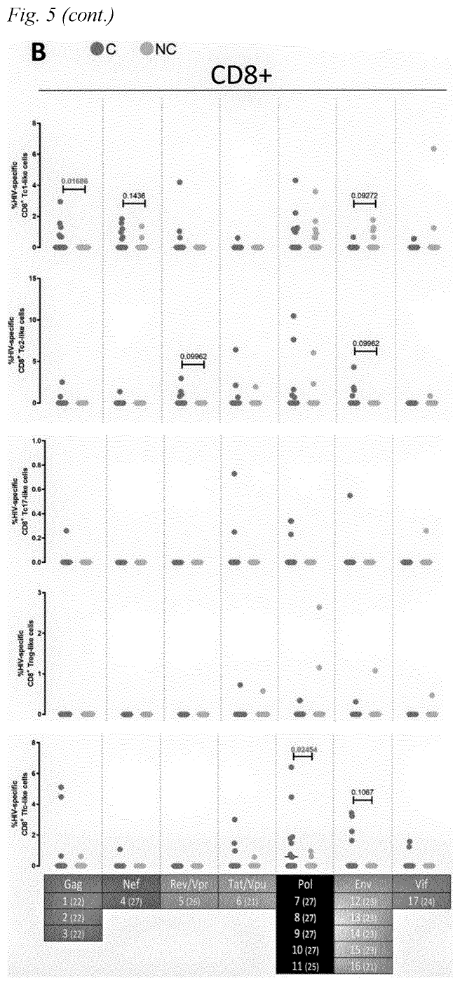

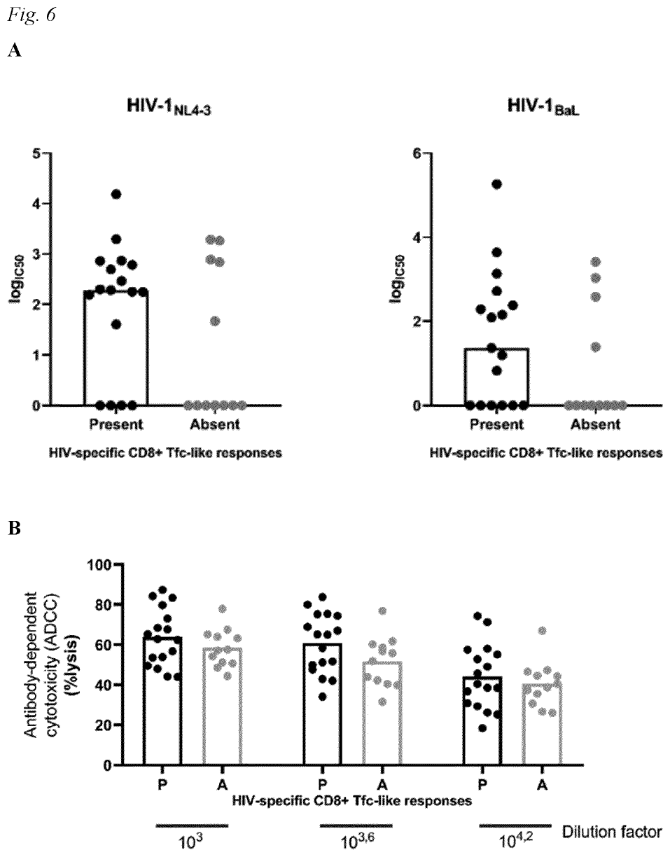

As the presence of CD8+ Tfc-like responses significantly differed between HIV controllers and non-controllers, the possibility that the acquisition of an anti-HIV CD8+ Tfc-like phenotype was related to a higher capacity of PBMCs to secrete T cell follicular-related cytokines (IL-4, IL-21) was explored. Total PBMCs (8 HIV+ controllers and 14 HIV+ non-controllers from the boosted flow study) were stimulated with the peptide pools that were found to trigger a Tfc-like responses in the initial boosted flow cytometry screening and the cytokine secretion of IFN-γ, IL-4 or IL-21 was assessed individually or in combination (IL-4 and IL-21) by single cytokine or boosted ELISPOT assay. While only 54% of the tested peptide pools induced IL-21 SFC and only 18% induced IL-4 SFC, the combined detection of IL-4 and IL-21 increased the sensitivity of the ELISPOT assay and responses against 72% of the pools were detected, in line with the boosted flow analysis results. Some responses that would be missed otherwise were able to be detected (FIG. 4). Interestingly, individuals who showed the presence of HIV-specific CD8+ Tfc-like responses showed a significantly higher innate capability to secrete IL-21, but not IL-4 in response to an unspecific stimulus (PHA, FIG. 7D).

T cell responses to different HIV proteins have been shown to exert different antiviral activity. This may be related to antigen presentation, viral sequence variability but also to differences in maturation and effector profile polarization of responses to different viral proteins (Shiner E, et al., PLoS One 2014; doi:10.1371/journal.pone.0100175.45). Indeed, the analysis of T-cell responders stratified by HIV-derived protein revealed marked differences in the polarization profiles of responding T-cell populations. See FIG. 8. Furthermore, these alterations were different in HIV controllers and non-controllers, thus linking viral protein specificity directly to the specific polarization of T cell effector functions and to in vivo natural virus control. In particular, and in line with previous studies, CD4+ T-cell responses to Envelope and of a Th1-like profile were more frequent in HIV+ non-controllers compared to controllers (χ² p=0.0536), while more CD4+ Tfh-like, Env-specific responders were seen in HIV+ controllers (χ² p=0.0772). HIV+ non-controllers also triggered a more prominent Pol-specific Treg-like response. See FIG. 8A. In the case of CD8+ T-cells, statistically significant differences were observed for the frequency of type-1 responses to Gag-reactive T-cell responses in HIV controllers (χ² p=0.0079), also in line with previous studies using single cytokine-based (IFN-γ) ELISPOT analyses (Zuñiga R, et al., J. Virol. 2006; 80). Controllers also showed a significantly higher number of Tc2-like responders upon Rev/Vpr or Env stimulation (χ² p=0.0374) and Tfc-like upon Pol and Env stimulation (χ² p=0.0271 and 0.0374, respectively. See FIG. 8B. When comparing the magnitude of responses, CD8+ Tc1-like Gag-specific (MW p=0.0169) and Tfc-like Pol-specific (MW p=0.0245) were stronger in controllers compared to non-controllers. Remarkably, accessory proteins (Rev, Vpr, Tat and Vpu) elicited alternative effector functions rather than type-1 responses.

Polyfunctional analysis performed using the SPICE v6 software revealed mostly T cell responses with unique T cell polarization profiles (i.e., not showing mixed polarization profiles), in line with responses having matured into distinct polarization profiles and strongly supporting the specificity of the employed boosted flow analyses. These results indicate that alternative HIV-specific T-cell profiling beyond conventional antiviral type-1 responses can identify novel T cell responses with protein-specific polarization that are associated with viral control. The data also demonstrate that CD8+ Tfc-like might play a particularly important role in such efficient anti-HIV immune response and extend prior observations to hitherto unrecognized T cell populations driving in vivo HIV control.

### Example 4: Circulating Type 2 and Follicular-Like T-Cells Show Different Memory Profiles in HIV+ Controllers and Non-Controllers

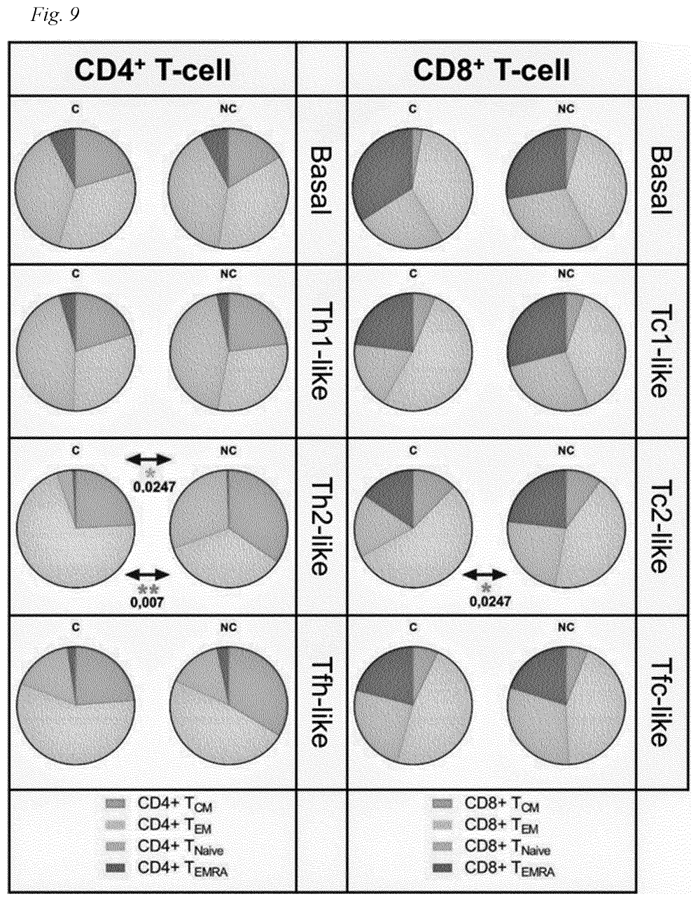

To determine whether the polarization of the anti-HIV T cell response and the evident control of in vivo virus control were associated with the induction of specific T cell differentiation and maturation, we determined CCR7 and CD45RA expression on these HIV-specific CD4+ and CD8+ type-1, type-2, and follicular-like T-cells. When comparing unstimulated total CD4+ and CD8+ basal memory phenotypes in HIV+ controllers versus non-controllers, no statistically significant differences were observed (median percentage of CD4 populations: TCM 18%, TEM 34.5%, TEMRA 7.5% and TNaive 40%; CD8+ T-cells: TCM 3%, TEM 37.8%, TEMRA 29.9% and TNaive 29.3%). See FIG. 9. In contrast, HIV-specific T-cells showed marked differences for memory subsets in the CD4+ Th2-like and Tfh-like, but not Th1 polarized cells, with a higher proportion of Th2-like and Tfh-like cells showing a TCM and TEM phenotype. Of note, the levels of CD4+ TEM contributing to Th2-like polarization were significantly higher (χ² p=0.007) in HIV+ controllers, while T Naive were more frequent in non-controllers (χ²=0.0247). While differences in memory subset distribution were less evident for CD8+ T-cells, a significant increase of TEM Tc2 frequencies (χ²=0.0247) and a reduction of T Naive Tc2 populations were observed in HIV-specific CD8+ cells when comparing controllers to non-controllers. Thus, boosted flow cytometry spanning profiles not previously assessed by this method, identified new T cell populations directly related to virus control, with important implications for preventive HIV vaccine development and for the optimization and monitoring of therapeutic (vaccine-based) interventions.

### Example 5: Expression of Cell Surface Polarization Markers in Individuals Showing CD8+ Tc2- and Tfc-Like Responses

To further validate that the CD4+ and CD8+ type 2- and follicular-like, HIV-specific T cells identified by our boosted flow cytometry approach, expressed markers indicative of such polarized T cell populations, we determined expression of CRTH2 for Th2/Tc2 or CXCR5, PD-1 and ICOS for Tfh/Tfc.

An unsupervised analysis by X-Shift yielded a total of 45 clusters based on the expression of CD4, CD8, CCR7, CD45RA, CRTH2, CXCR5, PD-1 and ICOS and a heatmap was performed based on the level of expression (MFI) of each marker. The contribution of each cluster inside the CD3+ population was used to compare HIV controller vs non-controller status as a fold change. Clusters with a ±2-fold change of the contribution to the CD3+ population (%) were included in the analysis. Three different clusters, one corresponding to CD8+ T-cells and two corresponding to CD4+ were differently represented in the two groups. Of those, one corresponded to CD4+ Tfh cells expressing ICOS, CXCR5 and PD-1. The other two clusters (one CD4+ and one CD8+) expressed high levels of CXCR5 but lacked the expression of PD-1 and ICOS. The potential relationship between the presence of circulating CRTH2+ Th2/Tc2 and circulating CXCR5+ Tfh/Tfc populations and HIV control was analyzed by manual supervised gating. These analyses did not reveal any differences in the levels of CD4+ Th2 cells expressing CRTH2 or CD4+ Tfh cells expressing CXCR5, PD-1 and ICOS. In CD8+ T cells, no difference in CRTH2 expressing Th2 cells was found, however, CD8+ CXCR5+PD-1+ICOS+ T-cells were significantly elevated in HIV+ controllers and also in those individuals capable to elicit HIV-specific CD8+ Tfc-like cytokine responses as seen by the Boosted flow, further validating the suitability, sensitivity and specificity of the Boosted flow cytometric approach to differentiate different disease control in HIV infection and highlighting the potential of this approach to identify novel T cell responses implicated in in-vivo virus control.

### Example 6: Higher Levels of CD8+ Tfc-Like Cells in HIV+ Controllers are Associated with Maturation of the Humoral Immune Response and Antibody Effector Functions

CD4+ Tfh-like cells have been shown to be required for the induction of effective humoral immunity. We thus assessed whether virus-specific CD4+, but especially also CD8+ Tfc-like, cells were related to antibody isotype switching, neutralization capacity, and ADCC effector function in HIV infection. To this end, plasma titers of HIV-1 BaL and HIV-1 NL4-3 Env-specific IgA, IgM, and IgG responses were analyzed in 24 HIV+ controllers (C), 20 HIV+ non-controllers (NC) and 8 HIV−, of which 15 HIV+ controllers and 14 HIV+ non-controllers were also analyzed by boosted flow cytometry in parallel. Uncontrolled HIV infection was associated with increased median IgA (p=0.0515) and IgM titers (p=0.0009), while HIV+ controllers' plasma contained higher titers of IgG (p=0.0182). In line with this finding, IgG/IgM and IgG/IgA ratios, reflecting isotype class switching towards IgG, were elevated in HIV controllers.

Neutralization capacity and ADCC activity were determined in the same groups of HIV controllers and non-controllers. Neutralization capacity was assessed against a panel of 5 HIV-1 pseudovirus, including strains from tiers 1A (NL4-3), 1B (BaL.01) and 2 (398F1, TRO11 and AC10). Among HIV+ controllers, a significantly higher proportion of individuals (76%) were able to neutralize HIV-1 NL4-3 compared to HIV non-controllers (37%, χ² p=0.0137). A similar trend was observed for the neutralization of HIV-1 BaL.01 (68% C vs 42% NC, χ² p=0.0657). Neutralization breadth was also significantly increased in controllers compared to non-controllers (median: 40% and 20% respectively; p=0.0111). The titer of HIV-1 NL4-3 strain-specific nAb was also significantly elevated in controllers (p=0.0059) and a trend (p=0.0819) for neutralization of HIV-1 BaL was observed as well. Along with neutralization, more potent ADCC activity was observed in controllers' plasma compared to non-controllers, at least at the lowest dilutions tested (1:10³, 1:10^{3.6}, 1:10^{4.2}). Significant positive correlations between ADCC capacity (1:10^{3.6} dilution) and neutralization IC₅₀ for HIV-1 NL4-3 (r=0.4562, p=0.0019) and HIV-1 BaL (r=0.3461, p=0.0214) were also observed. These associations were driven by the levels of IgG in the plasma samples, as Env-specific IgG (but not other isotypes) titers were found to be positively correlated with log IC50 for HIV-1 NL4-3 (Spearman r rho=0.5919, p-value<0.0001) and HIV-1 BaL (Spearman r rho=0.6423, p-value<0.0001). In line with this, IgG/IgM ratios also correlated with the neutralization of HIV-1 NL4-3 (r=0.5573, p<0.0001) and HIV-1 BaL (r=0.3028, p=0.0458). A similar relation of the IgG/IgM ratio was seen with the ADCC capacity. Not only with the IgG/IgM ratio for the HIV-1 NL4-3 Env (r=0.2770, p=0.0687), which is the same as the HIV strain expressed by the NKR-24 ADCC target cells but also with the IgG/IgM ratio for the HIV-1 BaL Env (r=0.3799, p=0.0110).

To further explore a possible relationship between the humoral immune response with HIV control, antibody titers and functional responses were correlated with three clinical parameters (CD4 count, CD4/CD8 ratio and plasma viral load). Anti-HIV-1BaL IgM titers showed a negative correlation with CD4 count (Spearman r rho=−0.6352, p<0.0001), CD4/CD8 ratio (Spearman r rho=−0.5628, p<0.0001) and a positive correlation with plasma viral load (Spearman r rho=0.4177, p=0.0048), in line with the elevated IgM levels seen in HIV non-controllers. Furthermore, plasma viral load was also negatively correlated with IgG/IgM ratio (r=−0.3094, p=0.0410), neutralizing capacity against HIV-1 NL4-3 (r=−0.3989, p=0.0073), and ADCC activity (r=−0.4863, p=0.0008).

### Example 7: HIV-Specific CD8+ Tfc-Like Responses are Linked to Antibody Isotype Class Switching and Humoral Function

The experimental data above showed that HIV-specific CD8+ Tfc-like responses were elevated in HIV controllers and that isotype ratios and IgG titers were related to HIV control as well. In view of these results, we tested whether the presence of CD8+ Tfc-like cells was related to these beneficial markers of the humoral immune response.

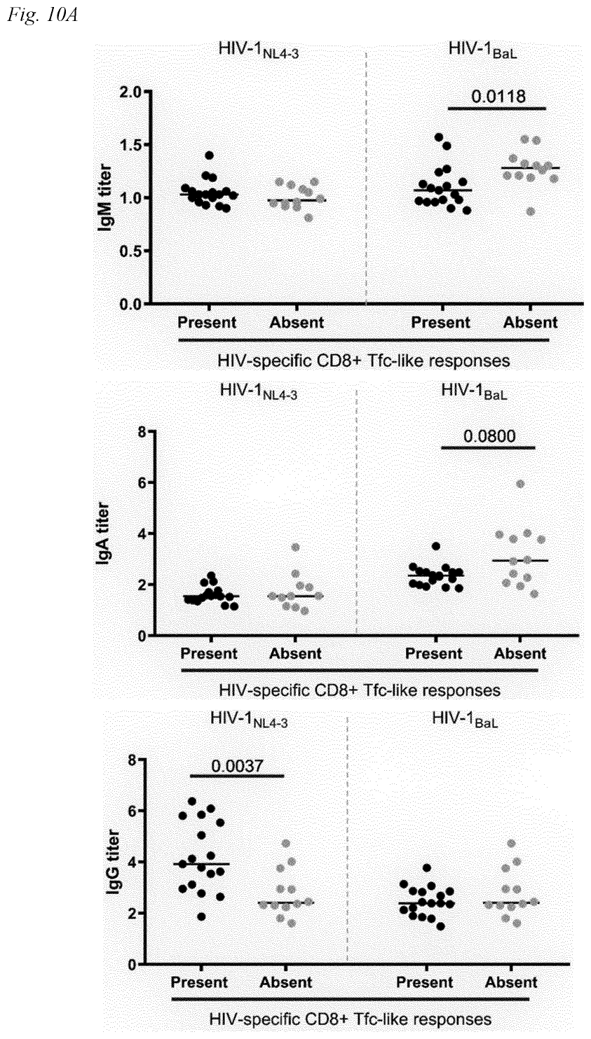

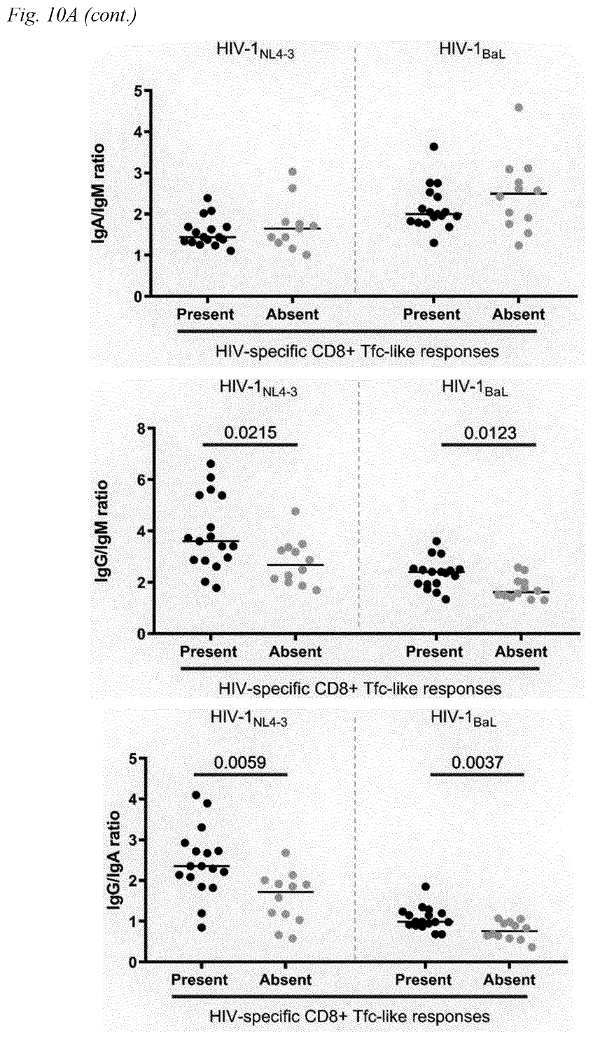

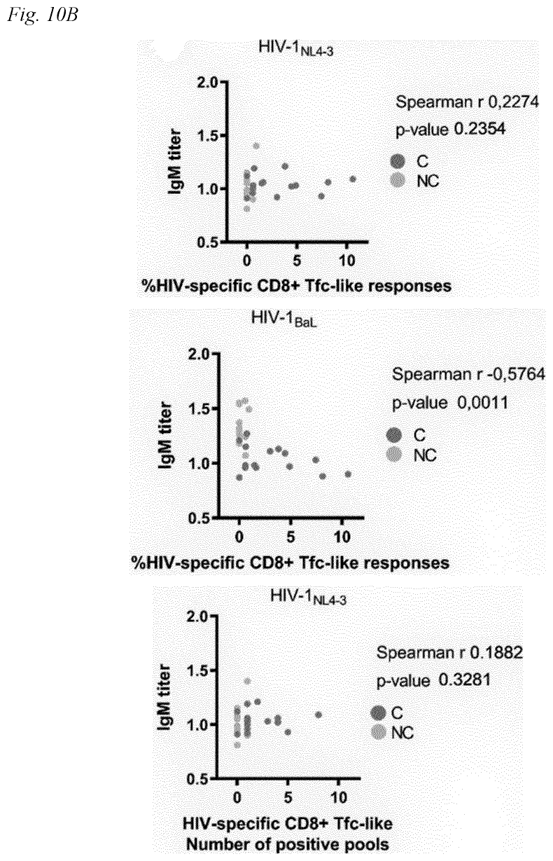

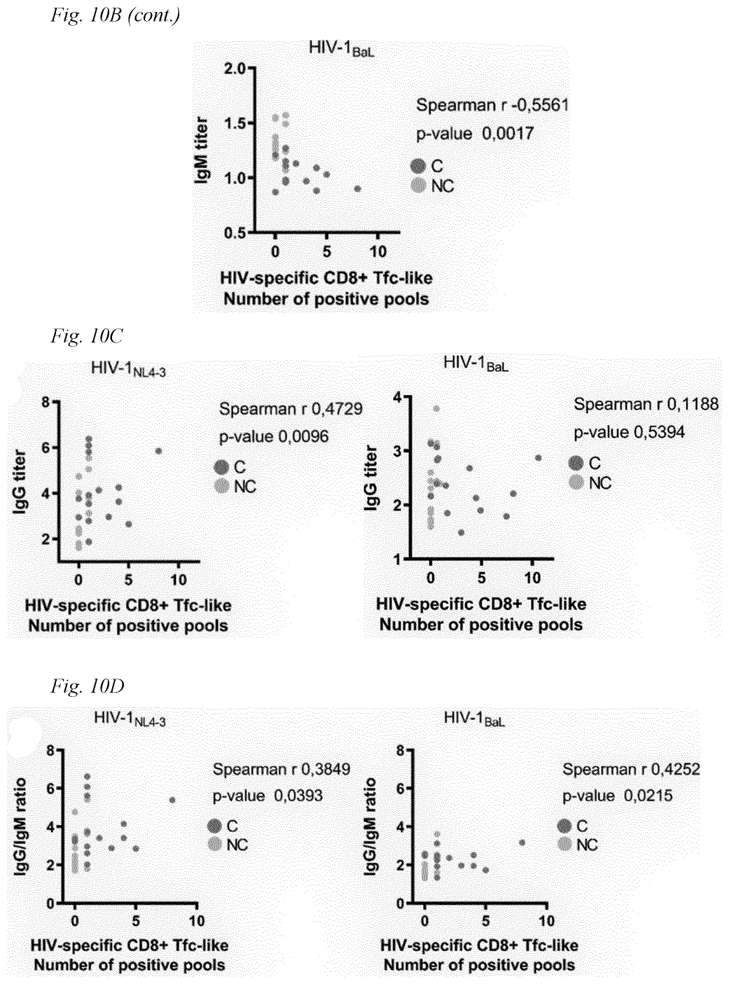

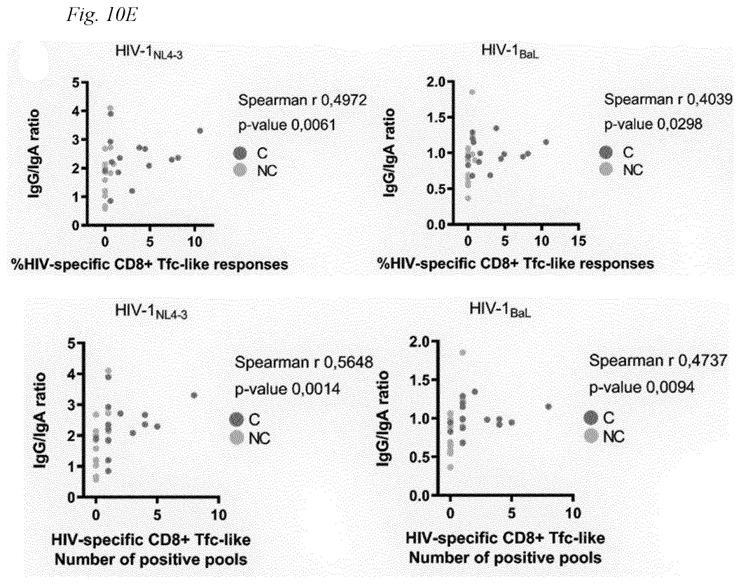

Plasma from individuals with detectable CD8+ Tfc-like cells demonstrated increased titers of IgG to HIV NL4-3 Env (p=0.0037) and decreased titers of IgM HIV-1 BaL Env (p=0.0118). This resulted in a significantly higher IgG/IgM ratio for both viral isolates (HIV-1 NL4-3 MW p=0.0215, HIV-1 BaL MW p=0.0123) and reflected increased antibody isotype class switching when compared to individuals with no detectable CD8+ Tfc-like responses. When measuring the balance of IgG with IgA levels, the results showed a clear bias towards IgG-mediated Ab activity when CD8+ Tfc-like responses were present (HIV-1 NL4-3 MW p=0.0059, HIV-1 BaL MW p=0.0037). See FIG. 10.

Finally, the association between CD8+ Tfc-like responses, isotype switching, and antibody-mediated effector function was analyzed by correlating CD8+ Tfc-like magnitude and breath with antibody titers, neutralization capacity, and ADCC activity. The magnitude and breadth of the CD8+ Tfc-like response correlated negatively with anti-HIV-1 BaL IgM (Magnitude: r=−0.5764, p-value=0.0011; Breadth: r=−0.5561, p-value=−0.0017, FIG. 10B) and positively with anti-HIV-1 NL4-3 IgG (Breadth: r=0.4729, p-value=0.0096, FIG. 10C) and IgG/IgM ratio for both strains (HIV-1 NL4-3: r=0.3849, p-value=0.0393; HIV-1 BaL: r=0.4252, p-value=0.0215, FIG. 10D). Interestingly, IgG/IgA ratio also correlated positively with Tfc-like magnitude and breadth, showing a preferential isotype switching towards IgG over IgA in those individuals who elicited this alternative, Tfc-like CD8+ effector functions. See FIG. 10E.

### REFERENCES

Adland, E. et al., PLoS One (2013) doi:10.1371/journal.pone.0073117.
Aid, M. et al., Front. Immunol. (2018) doi:10.3389/fimmu.2018.00895.
Annunziato, F. et al. J. Allergy Clin. Immunol. 135, 626-635 (2015).
Blanco, J. et al. J. Leukoc. Biol. (2004) doi:10.1189/jlb.0204100.
Chen, X. et al. Front. Immunol. (2018) doi:10.3389/fimmu.2018.02322.
Chung, A. W. et al. Viral Immunol. (2011) doi:10.1089/vim.2010.0108.
Clerici, M. & Shearer, G. M. Immunology Today vol. 14 (1993).
Côrtes, F. H. et al. in Journal of Acquired Immune Deficiency Syndromes (2015). doi:10.1097/QAI.0000000000000500.
Crotty, S. Cold Spring Harb. Perspect. Biol. 10, a032102 (2018).
de Taeye, S. W. et al. Front. Immunol. (2020) doi:10.3389/fimmu.2020.00740.
Fellay, J. et al. Science (80-.). (2007) doi:10.1126/science.1143767.
Fenoglio, D. et al. J. Allergy Clin. Immunol. 141, 2220-2233.e4 (2018).
Gillissen, M. A. et al. J. Immunol. Methods 434, (2016).
González, S. M. et al. Front. Immunol. 8, 1-9 (2017).
Graziosi, C. et al. Science (80-.). 265, (1994).
Guardo, A. C. et al. AIDS 29, (2015).
Hasenkrug, K. J. et al. PLoS Pathog. 14, 1-22 (2018).
Haynes, B. F. et al. N. Engl. J. Med. (2012) doi:10.1056/nejmoa1113425.
He, R. et al. Nature (2016) doi:10.1038/nature19317.
Hinks, T. S. C., Hoyle, R. D. & Gelfand, E. W. European Respiratory Review vol. 28 (2019).
Hur, E. M. et al. Blood (2012) doi:10.1182/blood-2012-04-422303.
Jacobs, E. S. et al. J. Virol. (2017) doi:10.1128/jvi.02051-16.
Johnson, S. et al. J. Virol. (2015) doi:10.1128/jvi.00438-15.
Jones, R. B. & Walker, B. D. Journal of Clinical Investigation (2016) doi:10.1172/JCI80566.
Joosten, S. A. & Ottenhoff, T. H. M. Hum. Immunol. 69, 760-770 (2008).
Kaufmann, D. E. et al., Comprehensive Analysis of Human Immunodeficiency Virus Type 1-Specific CD4 Responses Reveals Marked Immunodominance of gag and nef and the Presence of Broadly Recognized Peptides. J. Virol. (2004) doi:10.1128/jvi.78.9.4463-4477.2004.
Kiepiela, P. et al. Nat. Med. (2007) doi:10.1038/nm1520.
Kohler, S. L. et al. J. Immunol. (2016) doi:10.4049/jimmunol.1502174.
Koutsakos, M., Nguyen, T. H. O. & Kedzierska, K. J. Immunol. 202, 360-367 (2019).
Lee, N. et al. Am. J. Respir. Crit. Care Med. 190, (2014).
Locci, M. et al. Immunity 39, 758-769 (2013).
Lu, J. et al. J. Infect. Public Health 11, 685-690 (2018).
Martin-Gayo, E. et al. JCI Insight 2, 1-17 (2017).
Mascola, J. R. & Haynes, B. F. Immunol. Rev. (2013) doi:10.1111/imr.12075.
Mendoza, P. et al. Nature 561, (2018).
Miles, B. & Connick, E. T Trends in Microbiology (2016) doi:10.1016/j.tim.2016.02.006.
Molinos-Albert, L. M. et al. Sci. Rep. 7, (2017).
Mylvaganam, G. H. et al. Proc. Natl. Acad. Sci. U.S.A (2017) doi:10.1073/pnas.1621418114.
Overbaugh, J. & Morris, L. Cold Spring Harb. Perspect. Med. (2012) doi:10.1101/cshperspect.a007039.
Perdomo-Celis, F., Taborda, N. A. & Rugeles, M. T. Front. Immunol. 8, 1-13 (2017).
Pereyra, F. et al., J. Infect. Dis. (2008) doi:10.1086/526786.
Petrovas, C. et al. Sci. Transl. Med. (2017) doi:10.1126/scitranslmed.aag2285.
Planque, S. et al. AIDS (2010) doi:10.1097/QAD.0b013e3283376e88.
Ren, Y. et al. J. Virol. (2020) doi:10.1128/jvi.01808-20.
Roederer, M., Nozzi, J. L. & Nason, M. C. Cytom. Part A (2011) doi:10.1002/cyto.a.21015.
Romero-Martin, L et al. Front Immunol. 2022; 13: 928039.
Ruíz-Riol, M. et al. J. Infect. Dis. (2015) doi:10.1093/infdis/jiu534.
Sáez-Cirión, A. et al. J. Immunol. (2009) doi:10.4049/jimmunol.0803928.
Sallusto, F. Annu. Rev. Immunol. 34, 317-334 (2016).
Sauce, D., Gorochov, G. & Larsen, M. Sci. Rep. (2016) doi:10.1038/srep28129.
Schultz, B. T. et al. Immunity 44, 167-178 (2016).
Shiner, E. K., Holbrook, B. C. & Alexander-Miller, M. A. PLoS One (2014) doi:10.1371/journal.pone.0100175.
Song, W. & Craft, J. Immunol. Rev. 288, 85-96 (2019).
Su, B. et al. Frontiers in Immunology (2019) doi:10.3389/fimmu.2019.02968.
Valentine, K. M. & Hoyer, K. K. Frontiers in Immunology (2019) doi:10.3389/fimmu.2019.01322.
van Meijgaarden, K. E. et al. PLoS Pathog. 11, 1-24 (2015).
Walker, B. & McMichael, A. Cold Spring Harb. Perspect. Med. (2012) doi:10.1101/cshperspect.a007054.
Yu, D. & Ye, L. A Trends Immunol. 39, 965-979 (2018).
Zuñiga, R. et al. J. Virol. 80, (2006).

1-15. (canceled)

16. A method for assaying polarized T cell responses specific against an antigen in a subject which comprises:
(i) contacting a biological sample comprising T cells from a subject with a composition comprising the antigen and
(ii) determining the levels of a plurality of cytokines and/or chemokines produced by the T cells in the sample by boosted flow cytometry and/or boosted ELISPOT,
wherein a combination of antibodies against the plurality of cytokines and/or chemokines is used and the plurality of cytokines and/or chemokines is related to a specific T-cell polarization profile, wherein the antigen is at least one antigen from M. tuberculosis.

17. The method according to claim 16, wherein the antigen comprises at least one M. tb protein, preferably ESAT-6, CFP-10, or a combination thereof.

18. The method according to claim 16, wherein the plurality of cytokines and/or chemokines comprises cytokines and/or chemokines related to a Type 1 (Th1/Tc1), Type 2 (Th2/Tc2), Type 3 (Th17/Tc17), regulatory T-cell (Treg), and/or follicular T-cell (Tfh/Tfc) polarization profile, and/or other minor emergent polarization profiles.

19. The method according to claim 18, wherein the plurality of cytokines and/or chemokines comprises cytokines and/or chemokines related to a Type 1 (Th1/Tc1) and/or a Type 2 (Th2/Tc2) polarization profile.

20. The method according to claim 16, wherein the Type 1 (Th1/Tc1) plurality of cytokines comprises IFN-γ, IL-2, TNF-α, or a combination thereof.

21. The method according to claim 16, wherein the Type 2 (Th2/Tc2) plurality of cytokines comprises IL-4, IL-5, IL-10, IL-13, or a combination thereof.

22. The method according to claim 16, wherein the Type 3 (Th17/Tc17) plurality of cytokines comprises IL-17, IL-22, or a combination thereof.

23. The method according to claim 16, wherein the regulatory T-cell (Treg) plurality of cytokines comprises IL-10 and TGF-β, or a combination thereof.

24. The method according to claim 16, wherein the follicular T-cell (Tfh/Tfc) plurality of cytokines comprises IL-4, IL-21, CXCL-13 or a combination thereof.

25. The method according to claim 16, wherein the antibodies comprise monoclonal antibodies.

26. The method according to claim 16, wherein the sample to be analyzed is a peripheral blood cells preparation.

27. A kit comprising reagents for the detection of cytokines and/or chemokines according to claim 16.

28. A method for diagnosing tuberculosis comprising using the method of claim 16.

29. A method for diagnosing tuberculosis comprising using the kit of claim 27.

30. The method according to claim 28, for the diagnosis of active tuberculosis or latent tuberculosis.

31. The method according to claim 28, wherein the subject belongs to a population selected from children, elderly, immunocompromised patients, subject coinfected with HIV-1, subject under immunosuppressive treatment, and any combination thereof.
